(19) United States
(12) Patent Application Publication
(10) Pub. No.: US 2009/0326474 A1
(43) Pub. Date: Dec. 31, 2009

(54) UNIVERSAL STRAP DEVICE

(75) Inventors: Steven F. Bierman, Del Mar, CA (US); Richard A. Pluth, San DIego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Venetec International, Inc., Covington, GA (US)

(21) Appl. No.: 12/480,607

(22) Filed: Jun. 8, 2009

Related U.S. Application Data
(60) Provisional application No. 61/076,090, filed on Jun. 26, 2008.

(51) Int. Cl. A61M 5/32

(52) U.S. Cl. 604/180

(57) ABSTRACT

An anchoring system secures a medical article to the body of a patient and arrests movement of the catheter. The anchoring system includes an anchor pad that adheres to the patient's skin, a retainer supported by the anchor pad, and a strap attached to the retainer. The anchoring system can move between an open and a closed position. When in the open position, the retainer can receive a portion of the medical article and can be subsequently moved to the closed position. Advantageously, the anchoring system can receive medical articles of various sizes.

### CROSS-REFERENCE TO RELATED APPLICATIONS

This application claims the benefit under 35 U.S.C. §119(e) of U.S. Provisional Application No. 61/076,090, filed Jun. 26, 2008, which is hereby expressly incorporated by reference in its entirety.

### BACKGROUND

1. Field of the Invention

The present invention relates to a system for securing medical tubing to a patient.

2. Description of Related Art

Medical patients are often in need of repetitious administering of fluids or medications, or repetitious draining of fluids. It is very common in the medical industry to utilize medical tubing to provide various liquids or solutions to a patient. For example, medical tubing such as an intravenous (“IV”) catheter is often used to introduce fluids and medications directly into the bloodstream of a patient. In many cases, and particularly with respect to cardiac therapy, the IV catheter is introduced into a central or larger vein located close to the patient's heart. A typical catheter utilized in connection with a central vein is referred to as a “central venous catheter” (“CVC”). A venous catheter peripherally inserted into the central circulation through a vein in the arm is commonly referred to as a “peripherally inserted central catheter” (“PICC”). In these cases, long-term IV infusion typically requires that the medical tubing remain in place for many days. In some instances, a medical article may be attached to a patient for a lengthy period of time, requiring minimal movement for proper functioning.

It is often advantageous to restrict the movement of the medical tube or article. A moving medical article may cause discomfort to the patient, restrict the administering of fluids or medications or the draining of fluids, cause infection, or become dislodged from the patient unintentionally. The medical provider may attempt to restrict movement of the medical article by securing the distal end of the medical article to the patient using tape. Medical providers commonly place long pieces of tape across the distal end of the medical article, often in a crisscross pattern, to secure the medical article distal end to the patient. This securement is intended to inhibit disconnection between the medical article and the patient or between two medical articles, such as between a catheter and a drainage tube, as well as to prevent the medical article from catching on other objects, such as on a bed rail.

Taped connections, however, often collect contaminants and dirt. Normal protocol therefore requires periodic tape changes in order to inhibit germ growth. Periodic tape changes may also be necessary when replacing or repositioning the medical article. Frequent tape changes, however, lead to another problem: excoriation of the patient's skin. In addition, valuable time is spent applying and reapplying the tape to secure the medical article. And medical providers often remove their gloves when taping because most find the taping procedure difficult and cumbersome when wearing gloves. Not only does this further lengthen the procedure, but it also may subject the medical provider to possible infection and increase the risk of needle-stick.

Further, the strongest adhesive for attachment to the medical article cannot be implemented on tape used to secure the medical article to the patient since that same adhesive would contact the patient's skin. Therefore, the strongest adhesive for attachment to the medical article cannot be used because removal of such adhesive may damage the patient's skin.

### SUMMARY

The systems and methods of the present invention have several features, no single one of which is solely responsible for its desirable attributes. Without limiting the scope of this invention as expressed by the claims which follow, its more prominent features will now be discussed briefly. After considering this discussion, and particularly after reading the section entitled “Detailed Description of Certain Embodiments” one will understand how the features of this invention provide several advantages over traditional catheter securement systems.

One aspect of the present invention is an anchoring system for securing a medical article to a body of a patient. The system includes an anchor pad having an upper surface and a lower surface, at least a portion of the lower surface having an adhesive surface for contacting the patient's skin. The system further includes a retainer supported by the anchor pad and comprises a channel. The channel is configured to accept a portion of the medical article and at least one flexible strap. The strap is configured to wrap around at least a portion of the medical article so as to releasably secure the medical article to the retainer.

Another aspect is a medical line securement system that includes a medical article and an anchor pad that has a lower adhesive surface configured to attach to an epidermal layer of a patient. The system further includes a retainer that has a generally concave portion opposite the anchor pad and at least two opposing straps joined at a central section. The central section has a lower side adhered to the generally concave portion. The at least two opposing straps and central section have an upper side configured to contact the medical article so as to inhibit its longitudinal movement.

Another aspect is a securement device configured for use with a medical article. The device includes a retainer configured to receive a medical article and has at least one opening and at least one strap disposed within the at least one opening and configured to releasably engage the medical article.

Another aspect is a method of securing a medical article to a patient. The method includes providing a medical article, providing a retainer having a channel, an opening, and at least one strap attached to said retainer, and positioning a portion of the medical article over the channel. The method further includes folding the at least one strap over the portion of the medical article such that the at least one strap covers the portion of the medical article, threading at least a portion of the folded strap through the opening, and releasably securing the threaded portion to another portion of the at least one strap.

### BRIEF DESCRIPTION OF THE DRAWINGS

The above mentioned and other features of the invention will now be described with reference to the drawings of several embodiments of the present anchoring system. The illustrated embodiments of the anchoring system are intended to illustrate, but not to limit the invention. The drawings contain the following figures:

FIG. 1 is a perspective view of an anchoring system in accordance with an embodiment of the present invention and shows an anchor pad, a retainer, and a strap.

FIG. 2 is a top view of the anchor pad from FIG. 1.

FIG. 3 is a perspective view of the retainer from FIG. 1.

FIG. 4 is a top view of the retainer of FIG. 3.

FIG. 5 is an end view of the retainer of FIG. 3

FIG. 6 is a side view of the retainer of FIG. 3.

FIG. 7 is a bottom view of the retainer of FIG. 3.

FIG. 8 is a cross-sectional view of the retainer, taken along the line 8-8 of FIG. 4.

FIG. 9 is another cross-sectional view of the retainer, taken along the line 9-9 of FIG. 4.

FIG. 10 is a top view of the strap from FIG. 1.

FIG. 11 is a bottom view of the strap of FIG. 10.

FIG. 12 is a cross-sectional view of the strap, taken along the line 12-12 of FIG. 11.

FIG. 13 is an exploded view of the anchoring system of FIG. 1 showing the strap positioned above the retainer.

FIG. 14 is a perspective view of the anchoring system of FIG. 13 with the strap assembled to the retainer and a medical article positioned above the anchoring system.

FIG. 15 is a perspective view of the anchoring system of FIG. 14 with the medical article disposed within a channel portion of the retainer.

FIG. 16 is a perspective view of the anchoring system of FIG. 15 with the strap secured over the medical article.

FIG. 17 is a cross-sectional view of the anchoring system with secured medical article, taken along line 17-17 of FIG. 16.

FIG. 18 is a perspective view of an anchoring system in accordance with another embodiment of the present invention and shows a retainer and a strap.

FIG. 19 is a perspective view of the retainer from FIG. 18.

FIG. 20 is a top view of the retainer from FIG. 19.

FIG. 21 is an end view of the retainer of FIG. 19.

FIG. 22 is a side view of the retainer of FIG. 19.

FIG. 23 is a bottom view of the retainer of FIG. 19.

FIG. 24 is a cross-sectional view of the retainer, taken along the line 24-24 of FIG. 20.

FIG. 25 is another cross-sectional view of the retainer, taken along the line 25-25 of FIG. 20.

FIG. 26 is a top view of the strap of FIG. 18.

FIG. 27 is a bottom view of the strap of FIG. 26.

FIG. 28 is a cross-sectional view of the strap, taken along the line 28-28 of FIG. 27.

FIG. 29 is an exploded view of the anchoring system of FIG. 18.

FIG. 30 is a perspective view of the anchoring system of FIG. 29 with the strap assembled to the retainer and a medical article positioned above the anchoring system.

FIG. 31 is a perspective view of the anchoring system of FIG. 30 with the medical article disposed within a channel portion of the retainer.

FIG. 32 is a perspective view of the anchoring system of FIG. 31 with the strap secured over the medical article.

FIG. 33A is a cross-sectional view of the anchoring system with secured medical article, taken along line 33A-33A of FIG. 32.

FIG. 33B is another cross-sectional view of the anchoring system with secured medical article, taken along line 33B-33B of FIG. 32.

FIG. 34 is a perspective view of an anchoring system in accordance with another embodiment of the present invention and shows a retainer and a strap.

FIG. 35 is a perspective view of the retainer from FIG. 34.

FIG. 36 is a top view of the retainer from FIG. 35.

FIG. 37 is an end view of the retainer from FIG. 35.

FIG. 38 is a side view of the retainer from FIG. 35, taken from the side nearest which the retainer is viewed from in FIG. 35.

FIG. 39 is another side view of the retainer of FIG. 35, taken from the side opposite which the retainer is viewed from in FIG. 35.

FIG. 40 is a bottom view of the retainer from FIG. 35.

FIG. 41 is a cross-sectional view of the retainer, taken along the line 41-41 of FIG. 36.

FIG. 42 is another cross-sectional view of the retainer, taken along the line 42-42 of FIG. 36.

FIG. 43 is yet another cross-sectional view of the retainer, taken along the line 43-43 of FIG. 36.

FIG. 44 is a perspective view of the anchoring system of FIG. 34 with a medical article positioned above the anchoring system.

FIG. 45 is a perspective view of the anchoring system of FIG. 44 with the medical article disposed within a channel portion of the retainer.

FIG. 46 is a perspective view of the anchoring system of FIG. 45 with the strap secured over the medical article.

FIG. 47 is a cross-sectional view of the anchoring system with secured medical article, taken along line 47-47 of FIG. 46.

FIG. 48 is a perspective view of an anchoring system in accordance with another embodiment of the present invention and shows a retainer and a strap.

FIG. 49 is a perspective view of the retainer from FIG. 48.

FIG. 50 is a top view of the retainer from FIG. 49.

FIG. 51 is an end view of the retainer from FIG. 49.

FIG. 52 is a side view of the retainer of FIG. 49, taken from the side nearest which the retainer is viewed from in FIG. 49.

FIG. 53 is another side view of the retainer of FIG. 49, taken from the side opposite which the retainer is viewed from in FIG. 49.

FIG. 54 is a bottom view of the retainer from FIG. 49.

FIG. 55 is a cross-sectional view of the retainer, taken along the line 55-55 of FIG. 50.

FIG. 56 is another cross-sectional view of the retainer, taken along the line 56-56 of FIG. 50.

FIG. 57 is yet another cross-sectional view of the retainer, taken along the line 57-57 of FIG. 50.

FIG. 58 is a perspective view of the anchoring system of FIG. 49 with a medical article positioned above the anchoring system.

FIG. 59 is a perspective view of the anchoring system of FIG. 68 with the medical article disposed within a channel portion of the retainer.

FIG. 60 is a perspective view of the anchoring system of FIG. 59 with the strap secured over the medical article.

FIG. 61 is a cross-sectional view of the anchoring system with secured medical article, taken along line 61-61 of FIG. 60.

FIG. 62 is a perspective view of an anchoring system in accordance with another embodiment of the present invention and shows a strap and a retainer that has an adhesive spot and a passageway for receiving the strap.

FIG. 63 is a cross-sectional view of the retainer of FIG. 62 without the strap, taken along the line 62-62.

FIG. 64 is a perspective view of an anchoring system in accordance with another embodiment of the present invention and shows a retainer that has a passageway for an integral strap.

FIG. 65 is a cross-sectional view of the retainer of FIG. 64, taken along the line 65-65.

FIG. 66 is a cross-sectional view of the anchoring system of FIG. 64 with a secured medical article, taken along the line 65-65.

### DETAILED DESCRIPTION OF CERTAIN EMBODIMENTS

The present embodiments of the medical article anchoring system may be utilized with a variety of types of medical articles. It will be understood by one of skill in this art, in light of the present disclosure, that the anchoring system and retainer disclosed herein can be successfully utilized in connection with types of medical articles that include fluid drainage and delivery tubes and electrical wires, in addition to a variety of different types of catheters or other medical articles. For example, but without limitation, the retainer disclosed herein can be configured to receive and secure central venous catheters, peripherally inserted central catheters, hemodialysis catheters, Foley catheters, surgical drainage tubes, feeding tubes, chest tubes, nasogastric tubes, and scopes, as well as electrical wires or cables connected to external or implanted electronic devices or sensors. One skilled in the art can also find additional applications for the devices and systems disclosed herein. Thus, the illustrations and descriptions of the anchoring system in connection with a medical article are merely exemplary of one possible application of the anchoring system.

The anchoring system described herein is especially adapted to arrest lateral and/or transverse movement of a medical article, as well as hold the medical article against the patient. The anchoring system accomplishes this without meaningfully impairing (i.e., substantially occluding) fluid flow through the medical catheter. As described below, retention mechanisms to accomplish this include, among others, the shape of the channel that retains a section of the medical article, and a retaining strap either aligned with or positioned within the channel.

The anchoring system releasably engages the medical article. This allows the medical article to be disconnected from the anchoring system, and from the patient, for any of a variety of known purposes. For instance, the medical provider may want to remove the medical article from the anchoring system to ease disconnection of two connected medical articles or to clean the patient. The disengagement of the medical article from the anchoring system, however, can be accomplished without removing the anchoring system from the patient. In addition, no part of the anchoring system is destroyed during disengagement of the anchoring system. In this way, the anchoring system can be reused. It is not limited to use for only one medical article, but can be used multiple times for the same medical article or for different medical articles. After disengagement of the medical article, the anchoring system is ready for re-engaging with the same or a different medical article. A detailed description of embodiments of an anchoring system, and its associated method of use, now follows.

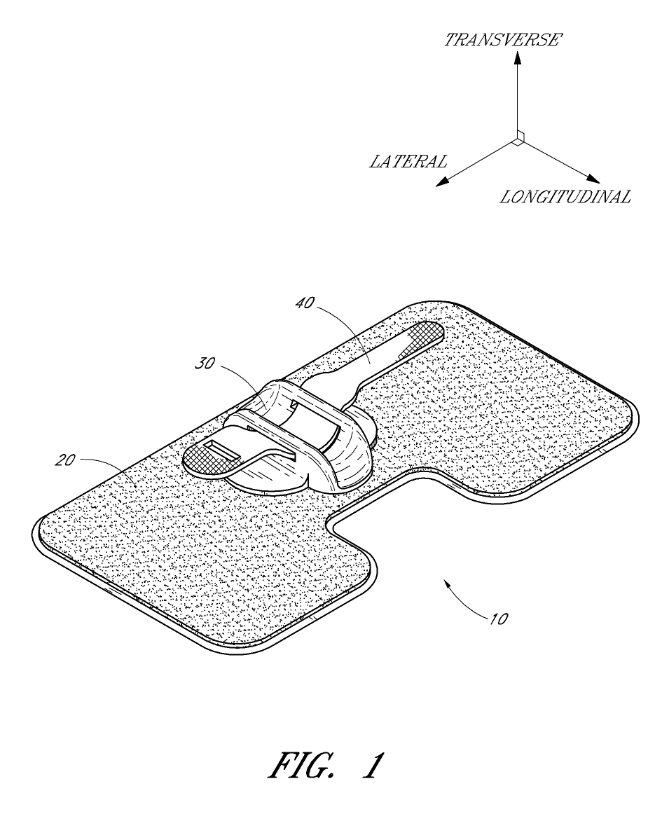

With reference now to FIG. 1, a first embodiment of an anchoring system 10 includes an anchor pad 20, a retainer 30, and a strap 40. The retainer 30 is attached to an upper surface of the anchor pad 20. The anchor pad 20 may in turn be secured to a patient's skin. The strap 40 may be detachably or permanently secured to the retainer 30, as will be described in more detail below, and is moveable between open and closed positions.

To assist in the description of the components of embodiments of the anchoring system, the following coordinate terms are used, consistent with the coordinate axes illustrated in FIG. 1. A “longitudinal axis” is generally parallel to a section of a medical article retained by the anchoring system 10. A “lateral axis” is normal to the longitudinal axis and is generally parallel to the plane of the anchor pad 20. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. Also, the terms “proximal” and “distal”, which are used to describe the present anchoring system 10, are used consistently with the description of the exemplary application. Thus, proximal and distal are used in reference to the center of the patient's body.

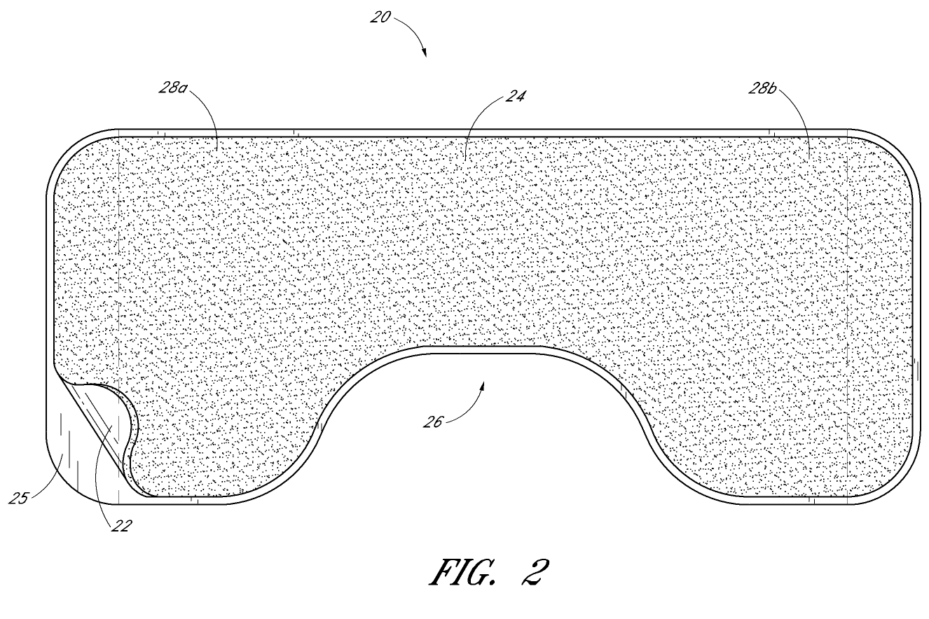

FIG. 2 illustrates the anchor pad 20. The anchor pad 20 has a lower adhesive surface 22 which may adhere to the skin of a patient and an upper surface 24 configured to support the retainer 30. In combination, the lower adhesive surface 22, upper surface 24, and possibly one or more intermediate layers may comprise a laminate structure. A suitable laminate that comprises a foam or woven material with an adhesive layer is available commercially from Avery Dennison of Painsville, Ohio.

The lower adhesive surface 22 may be a medical-grade adhesive and can be either diaphoretic or nondiaphoretic, depending upon the particular application. The lower adhesive surface 22 may have additional types of medical adhesives laminated thereto. Although not illustrated, it will be understood that the anchor pad 20 can include suture holes in addition to the adhesive layer to further secure the anchor pad 20 to the patient's skin.

The upper surface 24 may comprise a foam (e.g., closed-cell polyethylene foam) or woven material (e.g., tricot) layer. A surface of the foam or woven material layer constitutes the upper surface 24 of the anchor pad 20. In the alternative, the upper surface 24 may comprise an upper paper or other nonwoven cloth layer, and an inner foam layer may be placed between the upper surface 24 and lower adhesive surface 22.

A removable paper or plastic release liner 25 may cover the lower adhesive surface 22 before use. The liner may resist tearing and be divided into a plurality of pieces to ease attachment of the anchor pad 20 to a patient's skin. The liner may be made of a paper, polyester, or similar material.

In the illustrated embodiment, the anchor pad 20 has a concave section 26 that narrows the center of anchor pad 20 where the retainer 30 attaches. As a result, the lateral sides of anchor pad 20, illustrated as sections 28a and 28b, have more contact area which provides greater stability and adhesion to a patient's skin. The anchor pad 20, however, is not limited to requiring the concave section 26, as in the illustrated embodiment. The anchor pad 20 may have any shape that allows attachment of the anchor pad 20 to a patient's skin and allows the retainer 30 to attach to the anchor pad 20.

FIGS. 3-9 illustrate the retainer 30 without the anchor pad 20 and strap 40. Preferably, the retainer 30, anchor pad 20, and strap 40 are packaged and delivered to the medical provider in an assembled state. Of course the medical provider could assemble one or more of the components of the anchoring system 10. For example, the retainer 30 and strap 40 could be delivered in an unassembled state to the medical provider. The medical provider could then assemble the strap 40 to the retainer 30.

As can be seen in a perspective view of the retainer 30, as illustrated in FIG. 3, the retainer 30 comprises sidewalls 36a and 36b that define at least in part a channel 34 and wings 32a and 32b. The channel 34 is configured to accept a medical article. The sidewalls 36a and 36b include opening 38a and 38b, respectively. The openings 38a and 38b are configured to accept at least a portion of the strap 40, illustrated in FIG. 1. The retainer 30 also comprises curved declines 39a and 39b. A width and/or length of the wings 32a and 32b and/or channel 34 may be selected so as to allow a medical provider to more easily and naturally grip the retainer 30.

The wings 32a and 32b provide a greater contact area between the retainer 30 and the anchor pad 20 than the channel 34 would provide alone, allowing for a more secure attachment. The wings 32a and 32b may also provide stability for the retainer 30, minimizing the possibility that retainer 30 would roll if not secured to a stable surface. In one embodiment, the wings 32a and 32b may be omitted, such that only the channel 34 remains.

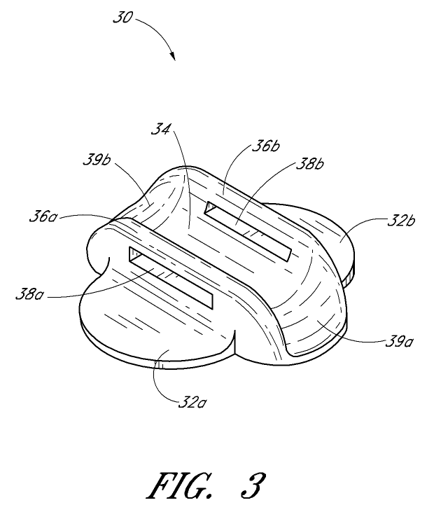

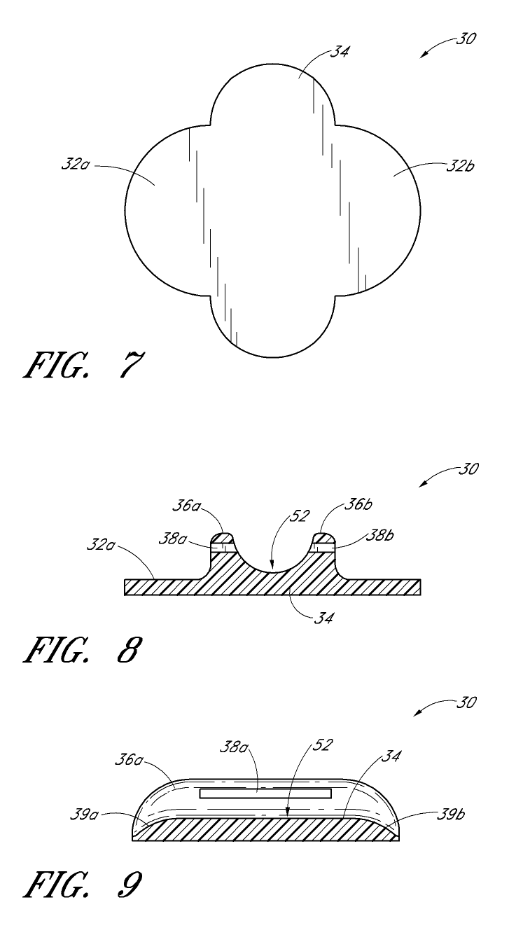

Although the wings 32a and 32b are illustrated as generally semicircular structures extending from the channel 34, they may be any shape, including square or triangular, and the wing 32a may be shaped different than the wing 32b. The shape of the wings 32a and 32b in the current embodiment can be seen in more detail in a top view of the retainer 30, illustrated in FIG. 4, and a bottom view of the retainer 30, illustrated in FIG. 7. The shape of the wings 32a and 32b may be selected to increase the security of attachment between the retainer 30 and the anchor pad 20, to ease manufacturing of the retainer 30, or to reduce the cost of manufacturing the retainer 30.

In the illustrated embodiment, an upper surface of the wings 32a and 32b is disposed so as to allow lateral access to the openings 38a and 38b. This disposition of the wings 32a and 32b provides space for a manufacturer to laterally slide the strap through the openings 38a and 38b.

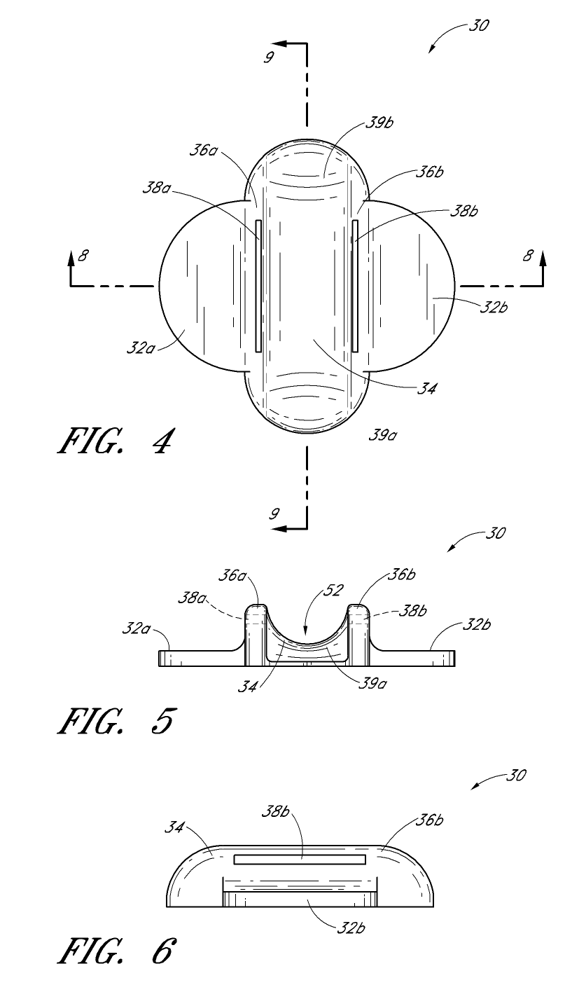

As can be seen in FIG. 5, the channel 34 comprises a concave surface 52 (which may also be described as a groove), and is configured to accept a medical article. The channel 34 is shaped such that a medical article can be placed therein, thereby resting at least partially within sidewalls 36a and 36b. When the medical article is placed in the channel 34, the medical article may rest on the concave surface 52. The channel 34 can be shaped in any way that allows the medical article to rest at least partially therein. In the illustrated embodiment, the concave surface 52 is generally semicircular. This can best be seen in an end view of the retainer 30, illustrated in FIG. 5, and a cross-section of the retainer 30, illustrated in FIG. 8. Such semicircular shape may roughly mirror the shape of a portion of a medical article that might be placed in the channel 34, thereby preventing excess lateral movement of the medical article.

As best seen in FIG. 4, the illustrated channel 34 is consistent in its width. However, in certain embodiments, the channel 34 varies in width along its longitudinal length. That is, in certain embodiments, the sidewalls 36a and 36b may diverge from each other in, for example, a generally linear or stepped manner from one longitudinal end of the retainer 30 to the other longitudinal end of the retainer 30, thereby varying the width of the channel 34. The channel 34 may have a tapering shape along at least a portion of the longitudinal axis. The channel 34 may also have a contoured shape to fit a specific medical article. For example, the channel 34 may be split into two channel portions, thereby forming a “y” shape, which may be used to retain a medical article such as a Foley catheter.

In the illustrated embodiment, the channel 34 is sized so that when the strap 40 is placed against the concave surface 52, a medical article placed on top of the strap 40 will still rest at least partially within the channel 34. The channel 34 is also wide enough to accept both a portion of the strap 40 and a portion of a medical article at the same time.

The longitudinal dimension of the channel 34 is sufficiently long to provide stability to the medical article along its length. That is, the longitudinal length of the portion of the medical article placed in the channel 34 and at least partially within the sidewalls 36a and 36b is sufficient to inhibit rocking of the medical article relative to the retainer 30 (i.e., to prevent the retainer 30 from acting as a fulcrum for the medical article).

The sidewalls 36a and 36b each contain an opening 38a and 38b, respectively. The size and shape of the openings 38a and 38b allow the strap 40 to pass through the sidewalls 36a and 36b. As can be seen in a cross-section taken along line 8-8 of FIG. 4, as is illustrated in FIG. 8, the openings 38a and 38b pass completely through the sidewalls 36a and 36b; thus, the strap 40 can pass from one side of the retainer 30 through the opening 38a, along the concave surface 52, and through the opening 38b to emerge from the other side of the retainer 30.

In the illustrated embodiment, the size and shape of the openings 38a and 38b roughly corresponds to a cross-section of the strap 40. Of course the openings 38a, 38b can be larger than the cross-section of the strap 40. The size and shape of the openings 38a and 38b can be seen in more detail in a cross-section of the retainer 30 taken along line 9-9 of FIG. 4, as illustrated in FIG. 9, and a side view of the retainer 30, as illustrated in Figure 6. Matching a cross-section of the strap 40 to the size and shape of the openings 38a and 38b minimizes longitudinal movement of the strap 40 when placed through the sidewalls 36a and 36b. In some embodiments, both openings 38a and 38b are a similar size and shape. In another embodiment, openings 38a and 38b may be sized or shaped differently and may be configured such that one end of the strap 40 may pass through both openings 38a and 38b, but the other end of the strap 40 may only pass through one of the openings, for example the opening 38a.

In the illustrated embodiment, the openings 38a and 38b are longitudinally centered along the retainer 30. Thus, the openings 38a and 38b are substantially aligned and the retainer 30 is substantially symmetrical. In another embodiment, the openings 38a and 38b may be located at a longitudinal location other than the center of the retainer 30, and the opening 38a may be located at a different longitudinal location than the opening 38b.

The channel 34 can comprise curved declines 39a and 39b. The bottom of the channel 34 is not flush with the top of the anchor pad 20. Curved declines 39a and 39b provide a transition from the channel 34 to an area level with the top of the anchor pad 20 or bottom of the retainer 30. It can be seen in a cross-section taken along line 9-9 of FIG. 4, as illustrated in FIG. 9, that the illustrated embodiment of the curved declines 39a and 39b is characterized by a gradual slope. Such gradual slope may be used to reduce the stress applied to a medical article secured to the retainer 30 and continuing at a distance from each end of the retainer 30. Other slopes, however, such as a more gradual slope or a sharper decline, may be used in place of the illustrated embodiment. Additionally, the curved declines 39a and 39b do not have to be identical or symmetrical. For example, the curved decline 39b may decline sharply to allow a medical article to be attached immediately thereafter to a patient, while the curved decline 39a may decline gradually to allow the medical article to comfortably continue a distance from the retainer 30.

The retainer 30 may be constructed as a single piece or from a plurality of different pieces. For example, the entire retainer 30 may be formed by injection molding, or the channel 34 and each wing 32a and 32b may be formed separately and thereafter joined together. The retainer 30 or portions thereof may be rigid or flexible. Suitable materials may include, for example, but without limitation, plastics, polymers or composites such as polypropylene, polyethylene, polycarbonate, polyvinylchloride, acrylonitrile butadiene styrene, nylon, olefin, acrylic, polyester, as well as moldable silicon, thermoplastic urethane, thermoplastic elastomers, thermoset plastics and the like. In one embodiment, the retainer 30 is formed by injection molding using a polyethylene or a polypropylene material or nylon. However, other materials can be utilized.

FIGS. 10-12 illustrate the strap 40. As illustrated in FIG. 10, the strap 40 comprises an upper surface 102 and an opening 104. The strap 40 is configured such that it may be slid through openings 38a and 38b of the retainer 30, illustrated in FIG. 3. The strap 40 is also configured to be wider at an area near where the opening 104 is located as compared with an area located opposite the opening 104. Thus, a width W1, measured at an area near where the opening 104 is located, is greater than a width W2, measured at an area opposite the opening 104. Such configuration allows the portion of the strap 40 located opposite the opening 104 (i.e., having width W2) to be inserted into and pass through the opening 104. Similarly, the opening 104 is of a size and shape that allows the portion of the strap 40 having width W2 to pass through the portion of the strap 40 having width W1. In the illustrated embodiment, the size and shape of the opening 104 roughly corresponds to a cross-section of the strap 40, which may minimize longitudinal movement of the strap 40 when placed through the opening 104.

The shape and construction of the strap 40 may otherwise be varied. Any number of shapes or designs of the strap 40 are possible and within the scope of this description. For example, the strap 40 may taper substantially uniformly from W1 to W2. Although the strap 40 is illustrated as a single piece of material, the strap 40 may also comprise several pieces of material attached together.

The ends of the strap 40 are illustrated as being rounded. Such rounded ends may facilitate placing the strap 40 through the openings 38a and 38b. Rounding the edges of the portion of the strap 40 located opposite the opening 104 may also facilitate placing such portion through the opening 104. Other embodiments include a strap with ends that are not rounded or with a single rounded end.

In one embodiment, the upper surface 102 comprises a material or coating that provides a high level of friction between the strap 40 and a medical article contacting the strap 40. Such material or coating may prevent the medical article from sliding across the upper surface 102. Hence, when the strap 40 is used in combination with the retainer 30 attached to the anchor pad 20 to secure the catheter, longitudinal movement of the medical article will be inhibited. For example, the upper surface 102 may comprise a rubber or textured fabric material. Alternatively, the upper surface 102 may comprise an adhesive material, such as one or more adhesive spots, described below in reference to FIGS. 62 and 63.

In another embodiment, the upper surface 102 comprises a material or coating that provides a low level of friction between the strap 40 and a medical article contacting the strap 40. Such material or coating allows the medical article to easily move across the upper surface 102. Hence, when the strap 40 is used in combination with the retainer 30 attached to the anchor pad 20 to secure the medical article, the medical article will remain secured to a patient, while still being able to slide across strap 40 and thus allowing increased movement by the patient. For example, the upper surface 102 may comprise a silicon or nylon material.

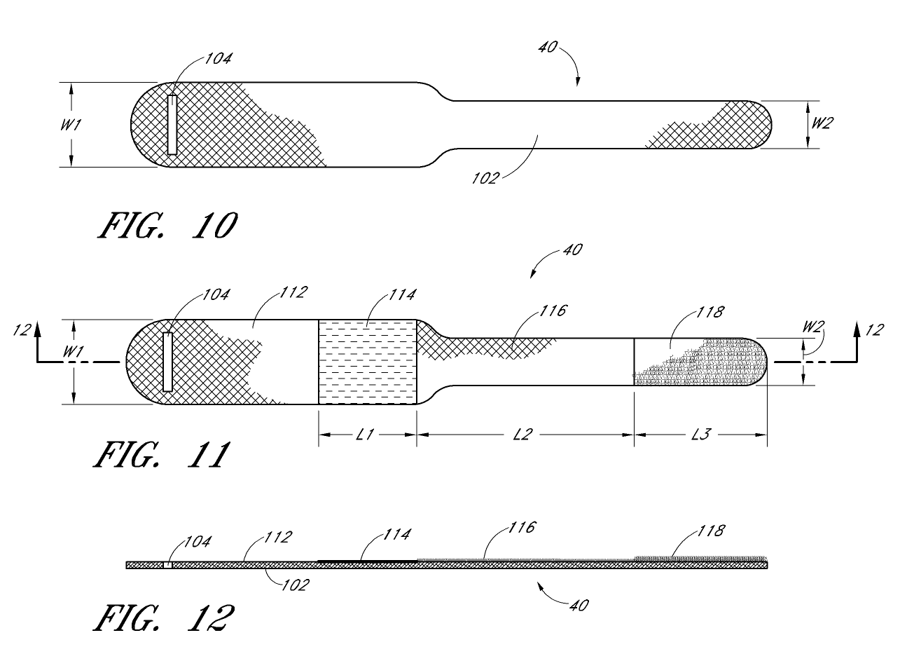

Viewed from the bottom, as illustrated in FIG. 11, strap 40 is comprised of a lower surface 112, adhesive 114, hook portion 116, and loop portion 118. In one embodiment, the adhesive 114, hook portion 116, and loop portion 118 are attached onto the lower surface 112, as illustrated in FIG. 12. In another embodiment, the lower surface 112 may only extend to the beginning of the adhesive 114, hook portion 116, or loop portion 118, and the remaining portions may be attached to each other laterally instead of placed on the lower surface 112. Thus, the strap 40 may be comprised of multiple sections or portions attached together. The lower surface 112 may be integral to the upper surface 102, illustrated in FIG. 10, or the lower surface 112 may be separate from and stacked on or laminated to the upper surface 102.

The adhesive 114 is configured to attach to the channel 34 of the retainer 30, illustrated in FIG. 3. In one embodiment, the adhesive 114 is a rectangular shape spanning the entire width W1 of the strap 40. In another embodiment, the adhesive 114 may be a different shape or may only partially cover the width of the strap 40. The adhesive may be a length L1 that is less than or approximately equal to a width of the concave surface 52 of the retainer 30, illustrated in FIG. 5. Such length will allow the entire length of the adhesive 114 to be attached to the retainer 30 without interfering with the openings 38a and 38b or other portions of the retainer 30. The adhesive 114 may comprise any adhesive that will attach the strap 40 to the retainer 30, such as pressure sensitive adhesives including acrylic and methacrylate adhesives, rubber-based pressure sensitive adhesives, certain polymers or copolymers (e.g., styrene copolymers, SIS/SBS), and silicones. The adhesive 114 may be an adhesive that will substantially permanently bond to the retainer 30, thereby promoting continued attachment of the strap 40 to the retainer 30. The adhesive 114 may be an adhesive that can be temporarily bonded to the retainer 30, thereby allowing removal of the strap 40 from the retainer 30. Alternatively, the adhesive 114 may be omitted.

The hook portion 116 and loop portion 118 are situated next to the adhesive 114. Either the hook portion 116 or loop portion 118 may be located next to the adhesive 114. FIG. 11 illustrates the hook portion 116 as being located next to the adhesive 114, but the placement of the hook portion 116 and loop portion 118 could be reversed, with the loop portion 118 being located next to the adhesive 114. The hook portion 116 and loop portion 118 may directly abut each other and/or the adhesive 114, as illustrated in FIG. 11, or there may be a distance between the portions and/or the adhesive 114.

The hook portion 116 and/or loop portion 118 can span the entire width of the strap 40. In another embodiment, the hook portion 116 and/or loop portion 118 may configured as various shapes or may only partially cover the width of the strap 40.

The hook portion 116 and loop portion 118 have a length L2 and L3, respectively, such that when the strap 40 is attached to the retainer 30 and drawn across a medical article placed in the retainer 30, at least a portion of the loop portion 118 can be passed through the opening 104 and folded back to contact at least a portion of the hook portion 116. More details regarding this procedure of attaching a medical article to the retainer 30 are provided below.

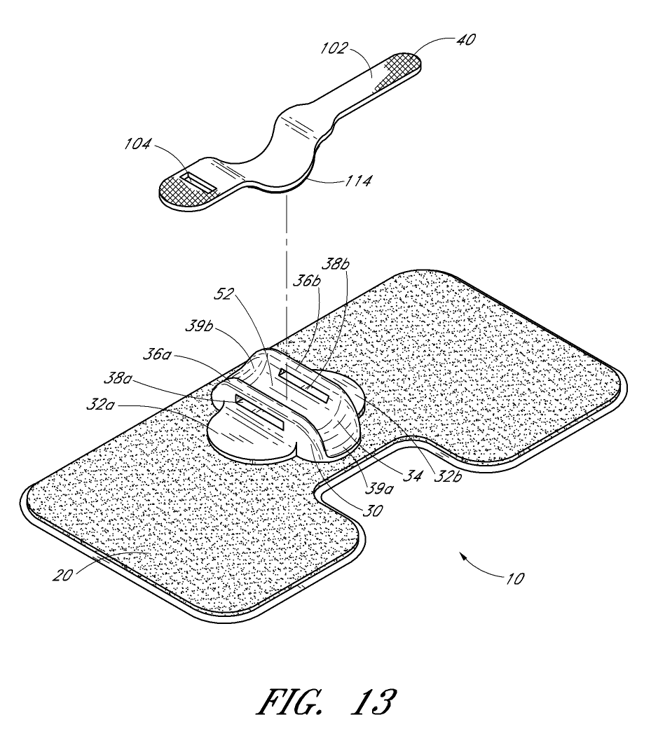

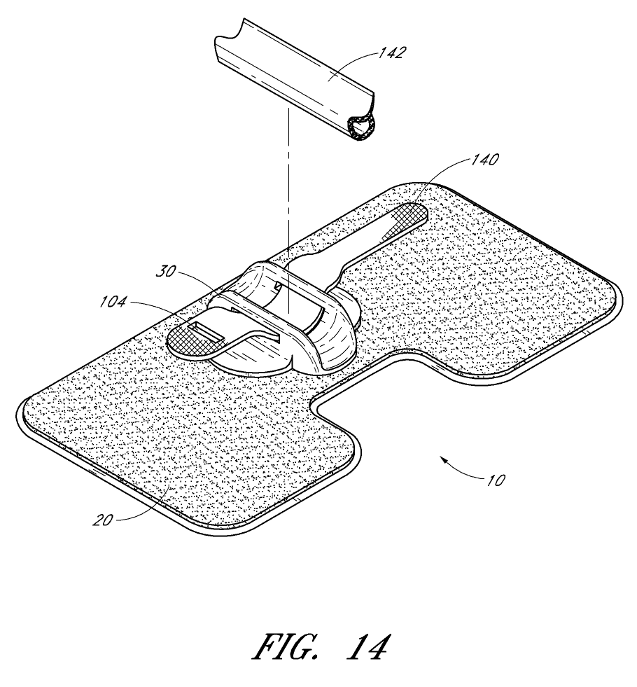

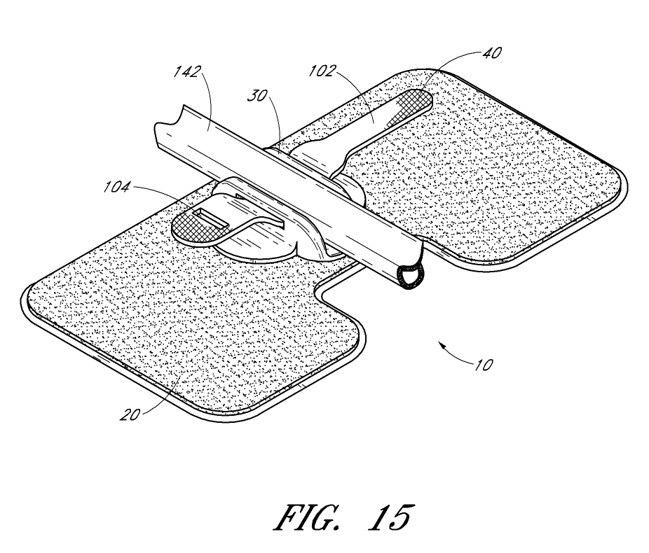

FIG. 13 illustrates the anchoring system 10 before the strap 40 has been attached to the retainer 30 or after the strap 40 has been removed from the retainer 30. The anchoring system 10 can be assembled by appropriately attaching or reattaching the strap 40 to the retainer 30, which is attached to the anchor pad 20. Preferably, the retainer 30, anchor pad 20, and strap 40 are packaged and delivered to the medical provider in an assembled state. Of course the medical provider could assemble one or more of the components of the anchoring system 10. For example, the retainer 30 and strap 40 could be delivered in an unassembled state to the medical provider. The medical provider could then assemble the strap 40 to the retainer 30. As can be seen in FIG. 13, the strap 40 may be preformed to follow a contour of the channel 34, such as the concave surface 52.

To attach the strap 40 to the retainer 30, a manufacturer or medical provider introduces either end of the strap 40 into opening 38a or 38b. If the strap 40 is introduced, for example, into opening 38a from a portion of the retainer 30 near the wing 32a, then the end of the strap 40 introduced into the opening 38a will pass through sidewall 36a and emerge in the center of the retainer 30, i.e., between the sidewalls 36a and 36b and near the concave surface 52. The medical provider may then introduce the end of the strap 40 into the opening 38b, whereby the end will pass through the sidewall 36b and emerge near the wing 32b. In this way, the strap 40 will pass from the wing 32a, through the channel 34, to the wing 32b, thereby attaching to the retainer 30, as shown initially in FIG. 1. The strap 40 thus is situated in a generally lateral direction, although other embodiments may include selecting the positions of the openings 38a and 38b and attaching the strap 40 to the retainer 30 so that the strap 40 is angled from a lateral direction.

If the strap 40 is introduced into opening 38a, for example, from a portion of the retainer 30 near the concave surface 52 (i.e., between the sidewalls 36a and 36b), then the end of the strap 40 introduced into the opening 38a will pass through the sidewall 36a and emerge near the wing 32a. The manufacturer or medical provider may then introduce the other end of the strap 40 into the opening 38b, whereby the end will pass through the sidewall 36b and emerge near the wing 32b. In this way, the strap 40 will pass from the wing 32a, through the channel 34, to wing 32b, thereby attaching to the retainer 30, as shown initially in FIG. 1. The strap 40 thus is situated in a generally lateral direction, although other embodiments may include selecting the positions of the openings 38a and 38b and attaching the strap 40 to the retainer 30 so that the strap 40 is angled from a lateral direction.

If the strap 40 comprises an adhesive 114, then the medical provider may pull the strap 40 through the retainer 30 such that the adhesive 114 is positioned between the sidewalls 36a and 36b and, for example, aligned with the concave surface 52. The medical provider may then press down on a portion of the strap 40 located opposite the adhesive 114 to cause contact between the adhesive 114 and the retainer 30. In this way, the adhesive 114 can be made to adhere to the retainer 30, providing a secure attachment of the strap 40 to the retainer 30 and minimizing movement of the strap 40 during operation of the anchoring system 10. To facilitate adhering the strap 40 to the retainer 30, a removable paper or plastic release liner may cover the adhesive 114. The medical provider may remove this release liner any time before pressing down on the strap 40, or it can be manufactured as such.

A medical article 142 can be placed in the retainer 30, as shown in FIGS. 14 through 16. The medical article 142 is initially placed above the anchoring system 10, as seen in FIG. 14. The medical provider can then lower the medical article 142 into the retainer 30. As seen in FIG. 15, the medical article 142 rests at least partially within the channel 34. In the illustrated embodiment, the medical article 142 is placed on top of the strap 40. In another embodiment, the medical article 142 may first be lowered into retainer 30 and then the strap 40 may be attached to the retainer 30, thereby placing the strap 40 over the medical article 142, or a free end of the medical article 142 may be inserted between the retainer 30 and the strap 40 if the strap 40 is already attached to the retainer 30. In either embodiment, the ends of the strap 40 project outward from the retainer 30 after the medical article 142 is placed into the retainer 30.

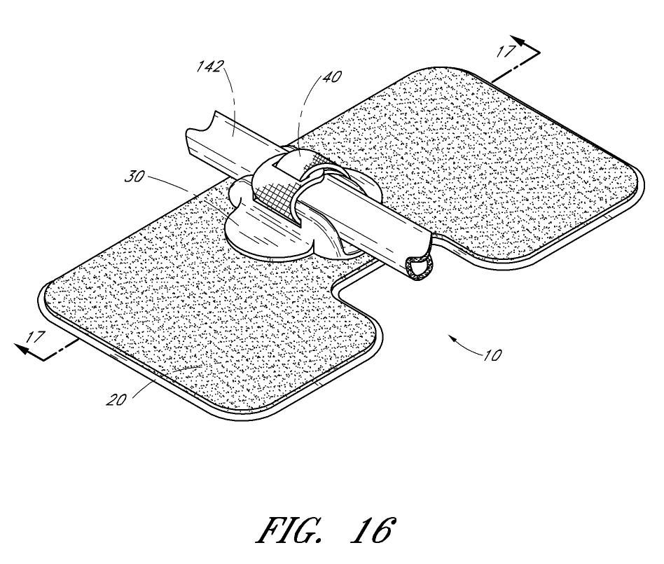

To secure the medical article 142 to the retainer 30, the medical provider places the end of the strap 40 located opposite the opening 104 across the medical article 142, and introduces the end into the opening 104. The medical provider draws the end through the opening 104, causing the upper surface 102 to contact the medical article 142 (in the embodiment where the medical article 142 is placed on top of the retainer 30 and the strap 40; if the medical article 142 is placed on top of the retainer 30, but below the strap 40, then the upper surface 102 will contact itself when the end is drawn through opening 104 and the medical article 142 will contact the lower surface 112). The medical provider then folds the strap 40 back in the opposite direction, pulling the strap 40 back towards the medical article 142 and causing the strap 40 to contact itself, as can be seen in FIG. 16. In this way, the medical article 142 can be secured to the retainer 30 and the anchoring system 10.

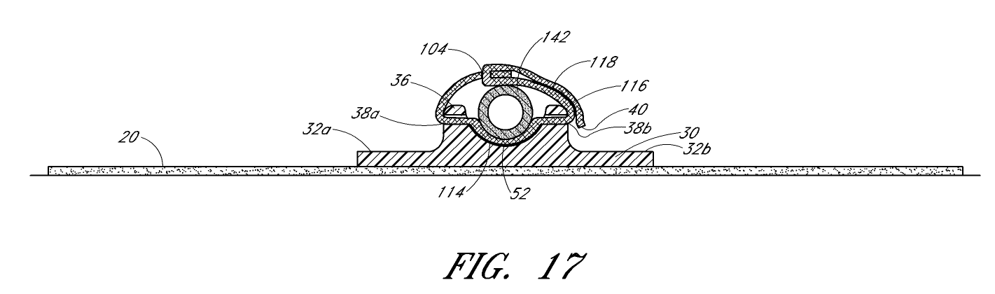

As can best be seen in FIG. 17, the strap 40 in the illustrated embodiment passes through the opening 38a and the sidewall 36a, along the concave section 52 and underneath the medical article 142, through the opening 38a and the sidewall 36a, over the top of the retainer 30 and the medical article 142, through the opening 104, and then back over the top of the retainer 30 and the medical article 142. Thus, the strap 40 wraps around the medical article 142 by passing through the retainer 30.

The strap 40 is secured to the retainer 30 by passing through the retainer 30 and may also be secured to the retainer 30 by the adhesive 114. Passing the strap 40 through the retainer 30 minimizes the chances of the strap 40 becoming unattached from the retainer 30. Adhering the strap 40 to the retainer 30 also reduces the chances of the strap 40 becoming unattached from the retainer 30, as well as minimizes movement of the strap 40 when the medical provider is securing the medical article 142.

When the strap 40 is folded back to contact itself, the hook portion 116 and loop portion 118 contact and attach to each other. This increases the likelihood that the strap 40 will stay in the position in which it has been placed by the medical provider. If the strap 40 has been drawn tightly across the medical article 142, the strap 40 will hold the medical article 142 substantially in place on the retainer 30. Therefore, the strap 40 in combination with the retainer 30 will inhibit movement of the medical article 142. In this way, the anchoring system 10 can be used to secure the medical article 142 on a patient's body.

Those of skill in the art will appreciate that attaching the medical article 142 to the retainer 30 and the anchoring system 10 in this way allows the use of medical articles of varying sizes. The end of the strap 40 opposite the opening 104 can be pulled through the opening 104 in varying lengths before folding the strap 40 back to contact itself. Thus, the size of an enclosed area created by the strap 40 or the strap 40 and the channel 34 can be increased or decreased as required. The size and location of the hook portion 116 and/or the loop portion 118 may be selected to facilitate the possibility of securing medical articles of varying size to the retainer 30.

The medical provider may separate the hook portion 116 from the loop portion 118 by pulling the end of the strap 140 opposite the opening 104. When sufficient force is applied to the end of the strap 40, the hook portion 116 and loop portion 118 will detach. After the detachment, the strap 40 may be loosened about the medical article 142 or the end of the strap 40 may be removed from the opening 104. This may allow the medical article 142 to be removed from the retainer 30. Such detachment, however, will not substantially impair the hook portion 116 and loop portion 118 from reattaching at a later time. Thus, the medical article 142 or a different catheter or device may be reattached to the retainer 30 at a later time. The advantages of being able to remove a medical article and reattach the same or a different medical article have already been described above.

After detaching the hook portion 116 and loop portion 118, the strap 40 may sometimes be removed from the retainer 30. If there is no adhesive 114 on the strap 40, then the medical provider can pull the strap 40 through the openings 38a and 38b, removing the strap 40 from the retainer 30. If there is an adhesive 114, then the strap 40 may be similarly removed if the adhesive 114 is not permanently bonded to the retainer 30. In this instance, the medical provider will first have to pull the strap 40 away from the retainer 30 so that the adhesive 114 detaches from the retainer 30. Then, the strap 40 may be removed from the retainer 30.

The strap 40 may be reattached to the retainer 30. The strap 40 may again be placed through the openings 38a and 38b to attach the strap 40 to the retainer 30. In the case of the adhesive 114 comprising reusable adhesives, the strap 40 may also be adhered to the retainer 30 again. Thus, not only is the medical article 142 detachable from, removable from, and able to be reattached to the retainer 30 and the anchoring system 10, the strap 40 may also be detached from, removed from, and reattached to the retainer 30.

The ability to remove the strap 40 may serve many purposes. For example, the medical provider may want to clean or sanitize the anchoring system 10. Removal of the strap 40 may facilitate this process. Additionally, the medical provider may want to replace the strap 40 with a different strap, such as when the strap 40 has become worn or when a different strap is more suited to a particular type of medical article or application.

The ability to reattach the strap 40 may serve many purposes as well. For example, the medical provider will not need to dispose of the strap 40 after every use. It may be possible to sanitize the strap 40 for a subsequent use. It may also be possible to exchange straps between different retainers, such as when a retainer has become worn or when a retainer having different characteristics is more beneficial for use with a particular type of medical article or for a particular application.

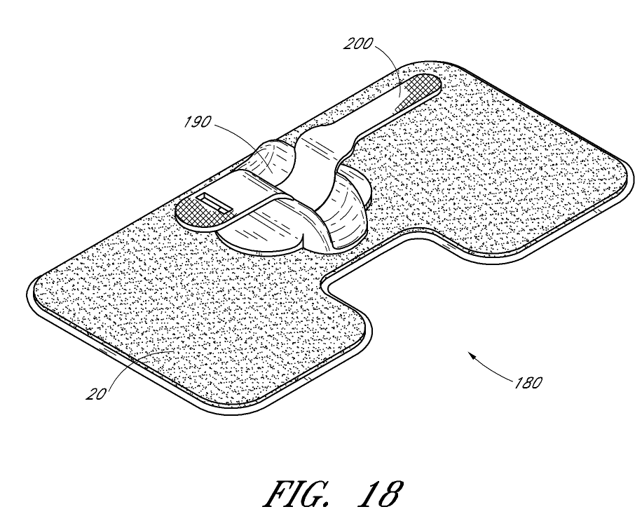

With reference now to FIG. 18, another embodiment of an anchoring system 180 includes an anchor pad 20, a retainer 190, and a strap 200. The anchoring system 180 is similar to the anchoring system illustrated in FIG. 1 except that the retainer 190 includes a recess 196 for the strap 200 and need not include openings 38a, 38b. The anchor pad 20 is the same as the anchor pad 20 illustrated in FIG. 1 and is configured to support the retainer 190. The strap 200 may be detachably or permanently secured to the retainer 190, as will be described in more detail below, and is moveable between open and closed positions.

FIGS. 19-25 illustrate the retainer 190. As can be seen in a perspective view of the retainer 190, as illustrated in FIG. 19, the retainer 190 comprises wings 192a and 192b, and a channel 194 configured to accept a medical article. The retainer 190 is configured to form a recess 196 in the retainer 190. The recess 196 is configured to accept the strap 200. The retainer 190 also comprises curved declines 199a and 199b. A width and/or length of the wings 192a and 192b and/or channel 194 may be selected so as to allow a medical provider to easily and naturally grip the retainer 190.

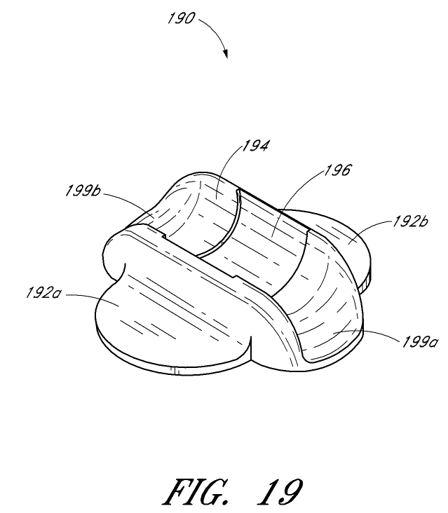

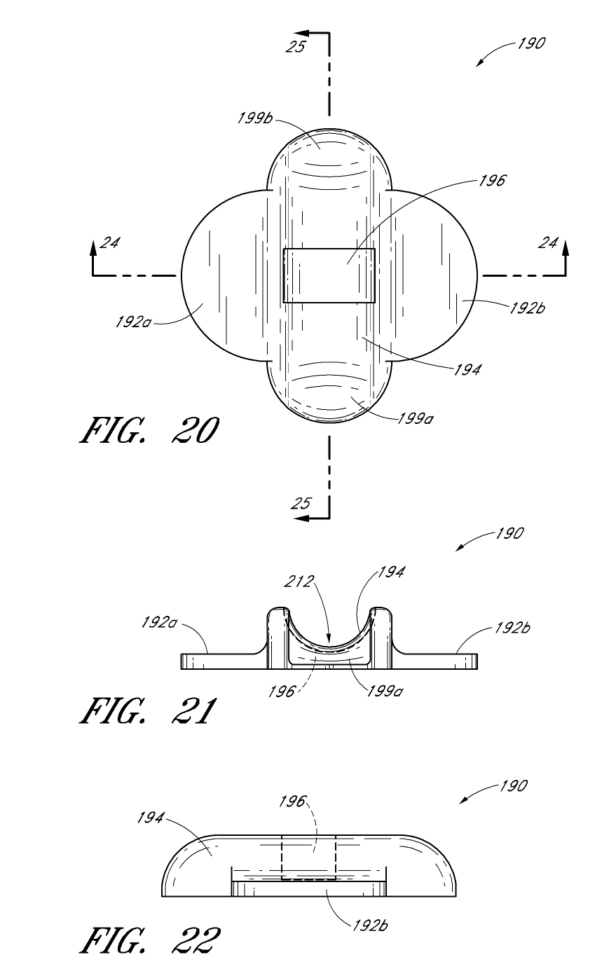

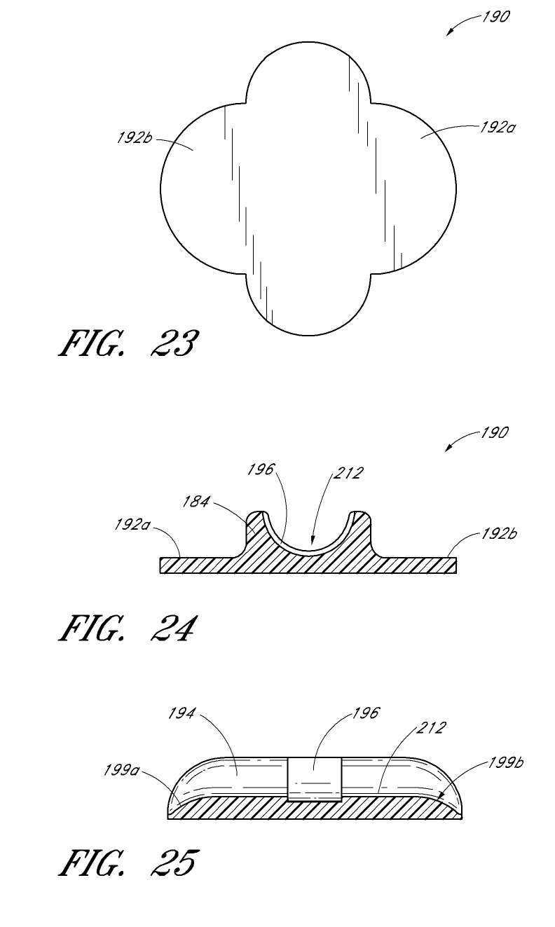

The wings 192a and 192b provide a greater contact area between the retainer 190 and the anchor pad 20 than the channel 194 would provide alone, allowing for a more secure attachment. The benefits and configurations of the illustrated wings 192a and 192b are similar those of wings 32a and 32b, described in reference to FIG. 3. An embodiment of the shape of the wings 192a and 192b is illustrated in FIGS. 20 and 23.

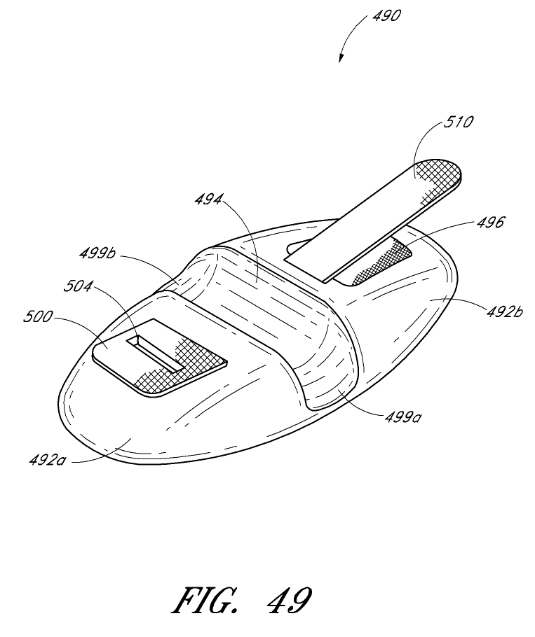

Although there are no openings in the retainer 190, such as the openings 38a and 38b in the retainer 30, the wings 192a and 192b may have a configuration similar to wings 32a and 32b. Thus, there may be enough space above the wings 192a and 192b for the fingers of a medical provider to manipulate the retainer 190 or portions of the strap 200. The wings 192a and 192b may also have a different configuration than wings 32a and 32b, as will be understood by one skilled in the art. An embodiment of one such different disposition is described below in reference to FIG. 49. An embodiment of the wing 192b is illustrated in FIG. 22.

The recess 196 is configured to accept the strap 200. Thus, a longitudinal width of the recess 196 is at least as wide as the strap 200. If the longitudinal width of the recess 196 is approximately equivalent to a width of the strap 200, longitudinal movement of the strap 200 can be minimized when the strap 200 is placed in the recess 196. A depth of the recess 196 may be selected according to an intended use of the retainer 190 or according to manufacturing concerns. In the illustrated embodiment, the depth of the recess 196 is approximately equivalent to a thickness of the strap 200. Hence, when the strap 200 is placed inside the recess 196, the strap 200 will be approximately flush with the retainer 190. In other embodiments, a depth of the recess 196 may be smaller or greater than a thickness of the strap 200, or a width and/or depth of the recess 196 may vary. An embodiment of the depth of the recess 196 can be seen in a cross section of the retainer 190 taken along a line 24-24 of FIG. 20.

As can be seen in FIG. 20, the recess 196 of the illustrated embodiment is situated in a generally lateral direction and is approximately longitudinally centered in the retainer 190. Other embodiments may include selecting the position of the recess 196 such that the recess 196 is angled from a lateral direction or is not longitudinally centered in the retainer 190.

As can be seen in FIG. 21, the channel 194 comprises a concave surface 212 (which may also be described as a groove), and is configured to accept a medical article. The channel 194 is shaped such that a medical article can be placed therein, thereby resting at least partially within the retainer 190. When the medical article is placed in the channel 194, the medical article may rest on the concave surface 212. The channel 194 can be shaped in any way that allows the medical article to rest at least partially therein, as described above in reference to the channel 34, illustrated in FIG. 5.

The channel 194 is sized so that when the strap 200 is placed in the recess 196, a medical article placed on top of the strap 200 will still rest at least partially within the channel 194. In the illustrated embodiment, the strap 200 fits into the recess 196 so as to be flush with the retainer 190.

In some embodiments, the longitudinal dimension of the channel 194 is sufficiently long to provide stability to the medical article along its length. That is, the longitudinal length of the portion of the medical article placed in the channel 194 is sufficient to inhibit rocking of the medical article relative to the retainer 190 (i.e., to prevent the retainer 190 from acting as a fulcrum for the medical article). The recess 196 is shaped and located so as to maintain this stability.

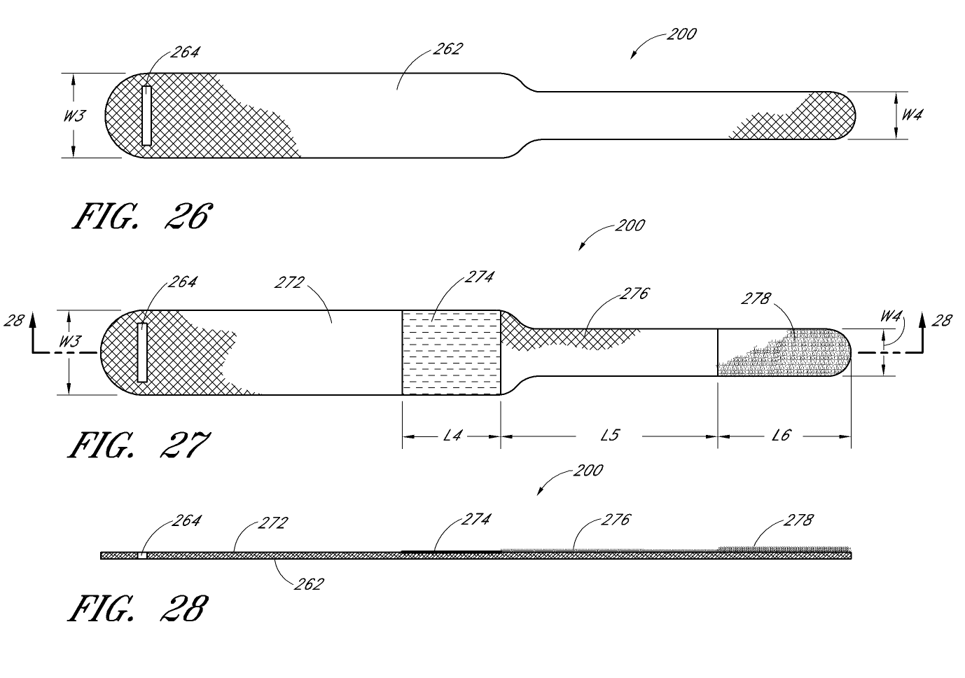

The channel 194 also comprises curved declines 199a and 199b. The channel 194 is not flush with the bottom of the retainer 190, but rather is removed from the bottom. Curved declines 199a and 199b provide a transition from the channel 194 to an area level with the bottom of the retainer 190, as described in reference to the curved declines 39a and 39b, illustrated in FIG. 9. An embodiment of the curved declines 199a and 199b is illustrated in FIG. 25.

The retainer 190 may be constructed as a single piece or from a plurality of different pieces, as described above in reference to the retainer 30, illustrated in FIG. 3. Suitable materials may vary, also as described in reference to the retainer 30.

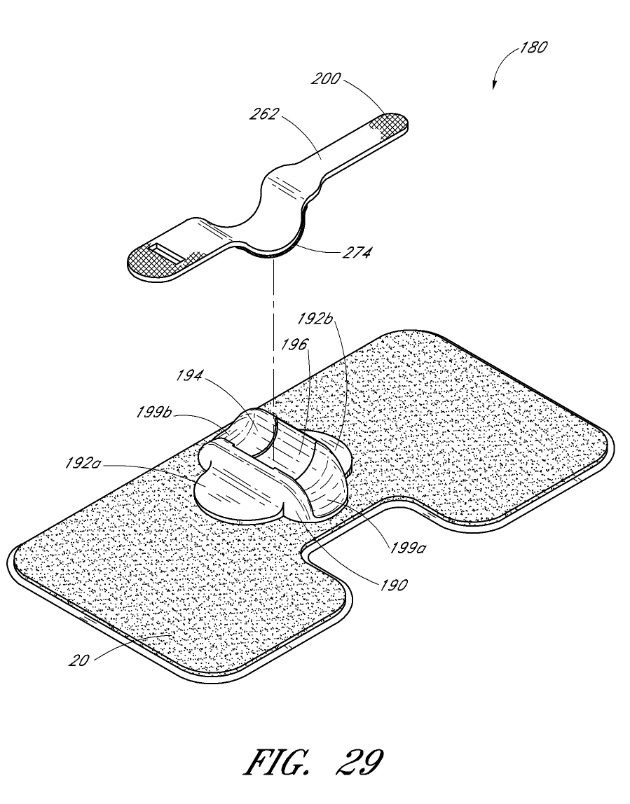

FIGS. 26-28 illustrate the strap 200. Viewed from the top, as illustrated in FIG. 26, the strap 200 is comprised of an upper surface 262. The strap 200 is configured to define an opening 264 in the strap 200. The strap 200 is configured such that it may be placed at least partially within the recess 196 of the retainer 190, illustrated in FIG. 19. The strap 200 is also configured to be wider at an area near where the opening 264 is located as compared with an area located opposite the opening 264. Thus, a width W3, measured at an area near where the opening 264 is located, is greater than a width W4, measured at an area opposite the opening 264. Such configuration allows the portion of the strap 200 located opposite the opening 264 (i.e., having width W4) to be inserted into and pass through the opening 264. Similarly, the opening 264 is of a size and shape that allows the portion of the strap 200 having width W4 to pass through the portion of the strap 200 having width W3. In the illustrated embodiment, the size and shape of the opening 264 roughly corresponds to a cross-section of the strap 200, which may minimize longitudinal movement of the strap 200 when placed through the opening 264.

The ends of the strap 200 are illustrated as being rounded. Rounding the edges of the portion of the strap 200 located opposite the opening 264 may facilitate placing such portion through the opening 264. Other embodiments include a strap with ends that are not rounded or with a single rounded end.

The shape and construction of the strap 200 may otherwise be varied, as described in reference to the strap 40, illustrated in FIG. 10. Any number of shapes, designs, materials, or constructions of the strap 200 are therefore possible and within the scope of this description.

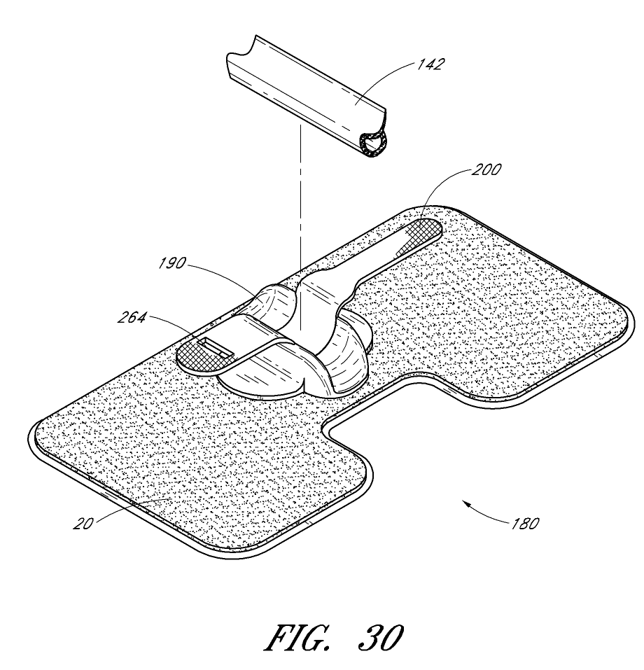

Viewed from the bottom, as illustrated in FIG. 27, strap 200 is comprised of a lower surface 272, adhesive 274, hook portion 276, and loop portion 278. In one embodiment, the adhesive 274, hook portion 276, and loop portion 278 are attached onto the lower surface 272, as illustrated in FIG. 28. In another embodiment, the lower surface 272 may only extend to the beginning of the adhesive 274, hook portion 276, or loop portion 278, and the remaining portions may be attached to each other laterally instead of placed on the lower surface 272. Thus, the strap 200 may be comprised of multiple sections or portions attached together. The lower surface 272 may be integral to the upper surface 262, illustrated in FIG. 26, or the lower surface 272 may be separate from and stacked on or laminated to the upper surface 262.

The adhesive 274 is configured to attach to an area of the retainer 190 within the recess 196, illustrated in FIG. 19. In one embodiment, the adhesive 274 is a rectangular shape spanning the entire width W3 of the strap 200. In another embodiment, the adhesive 274 may be a different shape or may only partially cover the width of the strap 200. The adhesive may be a length L4 that is less than or approximately equal to a lateral length of the recess 196. Such length of the adhesive 274 will allow the entire length of the adhesive 274 to be attached to the retainer 190 without interfering with other portions of the retainer 190. The adhesive 114 may comprise any adhesive that will releasably or permanently attach the strap 200 to the retainer 190, such as adhesives described in reference to the adhesive 114, illustrated in FIG. 1.

The hook portion 276 and loop portion 278 are situated next to the adhesive 274. The placement of the hook portion 276 and adhesive portion 278 in relation to each other and in relation to the adhesive is similar to the placement of the hook portion 116 and the loop portion 118 in relation to each other and in relation to the adhesive 114, described in reference to FIG. 11. The widths and shapes of the hook portion 276 and loop portion 278 are additionally similar to the widths and shapes of the hook portion 116 and the loop portion 118.

The hook portion 276 and loop portion 278 have a length L5 and L6, respectively, such that when the strap 200 is attached to the retainer 190 and drawn across a medical article placed in the retainer 190, at least part of the loop portion 278 can be passed through the opening 264 and folded back to contact at least a part of the hook portion 276. More details regarding this procedure of attaching a medical article to the retainer 190 will be provided below.

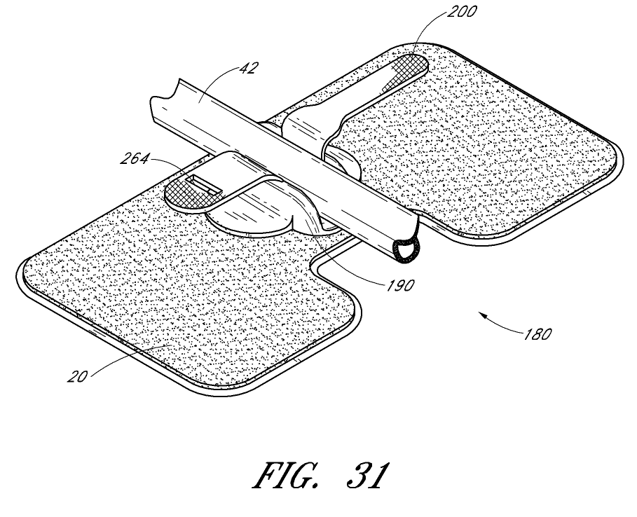

FIG. 29 illustrates the anchoring system 180 before the strap 200 has been attached to the retainer 190 or after the strap 200 has been removed from the retainer 190. Preferably, the retainer 190, anchor pad 20, and strap 200 are packaged and delivered to the medical provider in an assembled state. Of course the medical provider could assemble one or more of the components of the anchoring system 180. For example, the retainer 190 and strap 200 could be delivered in an unassembled state to the medical provider. The medical provider could then assemble the strap 200 to the retainer 190.

The anchoring system 180 can be assembled by appropriately attaching or reattaching the strap 200 to the retainer 190, which is attached to the anchor pad 20. As can be seen in FIG. 29, the strap 200 may be preformed to follow a contour of the channel 194, such as the concave surface 52 or a portion of the retainer 190 within the recess 196.

To attach the strap 200 to the retainer 190, a manufacturer or medical provider aligns at least a portion of the adhesive 274 with the recess 196. The medical provider then presses down on a portion of the strap 200 located opposite the adhesive 274 to cause contact between the adhesive 274 and the portion of the retainer 190 within the recess 196. In this way, the adhesive 274 can be made to adhere to the retainer 190, providing a secure attachment of the strap 200 to the retainer 190 and minimizing movement of the strap 200 during operation of the anchoring system 180. To facilitate adhering the strap 200 to the retainer 190, a removable paper or plastic release liner may cover the adhesive 274. The medical provider may remove this release liner any time before pressing down on the strap 200.

After the strap 200 is attached to the retainer 190, as can be seen in FIG. 30, the strap will extend laterally across the retainer 190. The ends of the strap 200 may extend beyond the channel 194 towards the wings 192a and 192b, and the ends of the strap 200 may rest on the wings 192a and 192b. Situating the strap 200 in this way will ease attachment of a medical article 142 to the retainer 190. As illustrated, the strap 200 may be situated in a generally lateral direction, although other embodiments may include selecting the position of the recess 196 and attaching the strap 200 to the retainer 190 so that the strap 200 is angled from a lateral direction.

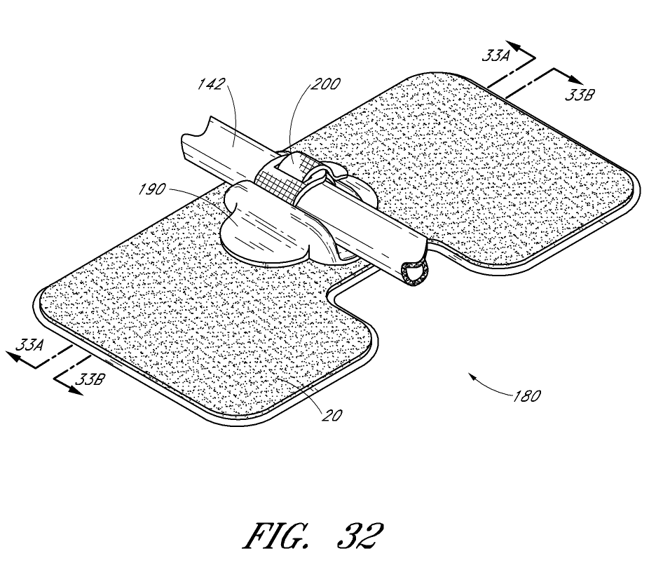

The medical article 142 can be placed in the retainer 190, as shown in FIGS. 30 through 32. The medical article 142 is placed above the anchoring system 180, as seen in FIG. 30. The medical provider can then lower the medical article 142 into the retainer 190. As seen in FIG. 31, the medical article 142 rests at least partially within the channel 194. The ends of the strap 200 project outward from the retainer 190 after the medical article 142 is placed into the retainer 190.

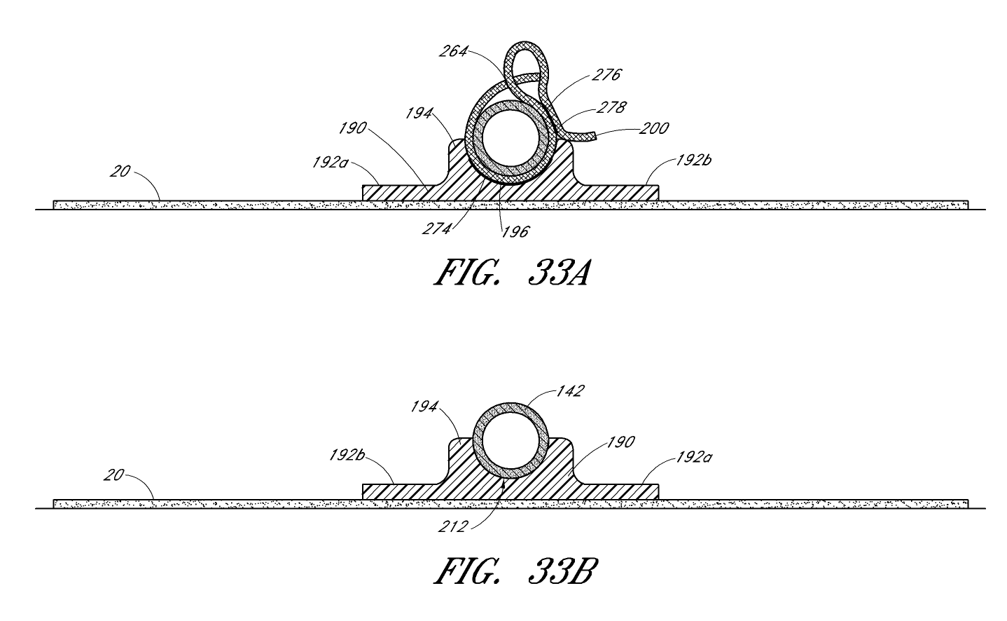

To secure the medical article 142 to the retainer 190, the medical provider places the end of the strap 200 located opposite the opening 264 across the medical article 142, and introduces the end into the opening 264. The medical provider draws the end through the opening 264, causing the upper surface 262 to contact the medical article 142. The medical provider then folds the strap 200 back in the opposite direction, pulling the strap 200 back towards the medical article 142 and causing the strap 200 to contact itself, as can be seen in FIG. 32. In this way, the medical article 142 can be secured to the retainer 190 and the anchoring system 180.

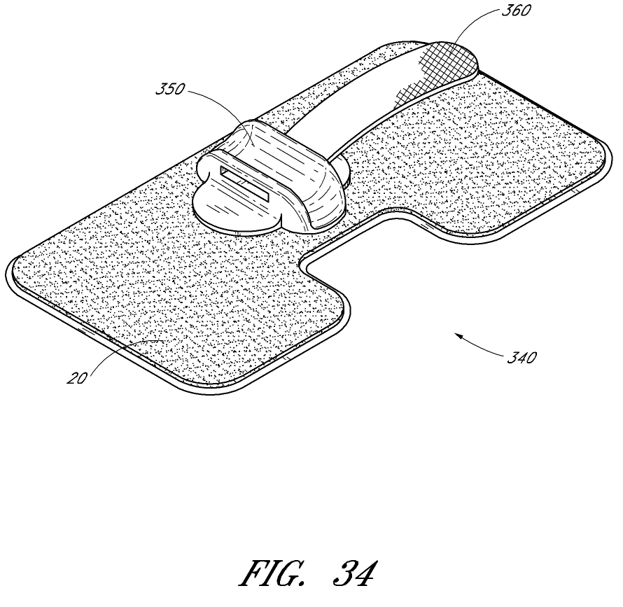

As can best be seen in FIG. 33, the strap 200 in the illustrated embodiment rests inside the recess 196, thereby being situated underneath the medical article 142. The strap 200 passes over the top of the medical article 142, through the opening 264, and then back over the top of the medical article 142, contacting itself and causing the hook portion 276 to attach to the loop portion 278. Thus, the strap 200 wraps around the medical article 142.

The strap 200 is secured to the retainer 190 by the adhesive 274. Adhering the strap 200 to the retainer 190 allows the attachment of the medical article 142 to the retainer 190 and minimizes movement of the strap 200 when the medical provider is securing the medical article 142.

When the strap 200 is folded back to contact itself, the hook portion 276 and loop portion 278 contact and attach to each other. This increases the likelihood that the strap 200 will stay in the position in which it has been placed by the medical provider. If the strap 200 has been drawn tightly across the medical article 142, the strap 200 will hold the medical article 142 substantially in place on the retainer 190. Therefore, the strap 200 in combination with the retainer 190 will inhibit movement of the medical article 142. In this way, the anchoring system 180 can be used to secure the medical article 142 on a patient's body.

Those of skill in the art will appreciate that attaching the medical article 142 to the retainer 190 and the anchoring system 180 in this way allows the use of medical articles of varying sizes. The end of the strap 200 opposite the opening 264 can be pulled through the opening 264 in varying lengths before folding the strap 200 back to contact itself Thus, the size of an enclosed area created by the strap 200 can be increased or decreased as required. The size and location of the hook portion 276 and/or the loop portion 278 may be selected to facilitate the possibility of securing medical articles of varying size to the retainer 190.

The medical provider may separate the hook portion 276 from the loop portion 278 by pulling the end of the strap 200 opposite the opening 264. When sufficient force is applied to the end of the strap 200, the hook portion 276 and loop portion 278 will detach. After the detachment, the strap 200 may be loosened about the medical article 142. This may allow the medical article 142 to be removed from the retainer 190. Such detachment, however, will not substantially impair the hook portion 276 and loop portion 278 from reattaching at a later time. Thus, the medical article 142 or a different catheter or device may be reattached to the retainer 190 at a later time. The advantages of being able to remove a medical article and reattach the same or a different medical article have already been described above.

After detaching the hook portion 276 and loop portion 278, the strap 200 may sometimes be removed from the retainer 190. If the adhesive 274 has not permanently bonded to the retainer 190, the medical provider can pull the strap 200 away from the retainer 190 so that the adhesive 274 detaches from the retainer 190, thereby allowing removal of the strap 200 the retainer 190.

The strap 200 may also sometimes be reattached to the retainer 190. In the case of the adhesive 274 comprising reusable adhesives, the strap 200 may be adhered to the retainer 190 again. Thus, not only is the medical article 142 detachable from, removable from, and able to be reattached to the retainer 190 and the anchoring system 180, the strap 200 may also be detached from, removed from, and reattached to the retainer 190.

The ability to remove and reattach the strap 200 from the retainer 190 may serve many purposes, as described above.

In the illustrated embodiment, the medical article 142 rests on the strap 200, as can be seen in FIG. 33. As described above, a depth of the recess 196 is approximately equivalent to a thickness of the strap 200. Thus, the top of the strap 200 will be approximately flush with the concave surface 212 when placed inside the retainer. Hence, the medical article 212 will not only rest on the strap 200 when the medical article is placed inside the retainer 190, but the medical article will also rest on the concave surface 212. This can best be seen in a cross-section taken along line 33B of FIG. 32, illustrated in FIG. 33B.

In the illustrated embodiment, transverse movement of the medical article 142 will be inhibited when the medical article 142 is attached to the retainer 190 by the strap 200. When a depth of the recess 196 is approximately equivalent to a thickness of the strap 200, the strap 200 will not act like a fulcrum. The portions of the medical article that are within the retainer 190, but not resting on the strap 200, will be less likely to move transversely. In addition, the portion of the medical article 142 passing over the recess 196 will be less likely to move transversely if that portion rests on the strap 200 instead of there being a gap between the strap 200 and the medical article 142, as there may be if a depth of the recess 196 is not equivalent to a thickness of the strap 200.

With reference now to FIG. 34, another embodiment of an anchoring system 340 includes an anchor pad 20, a retainer 350, and a strap 360. The retainer 350 is attached to an upper surface of the anchor pad 20. The anchor pad 20 may in turn be secured to a patient's skin. The strap 360 is attached to the retainer 350. The strap 360 is moveable between open and closed positions. The anchor pad 20 is the same as the anchor pad 20 illustrated in FIG. 2 and is configured to support the retainer 340.

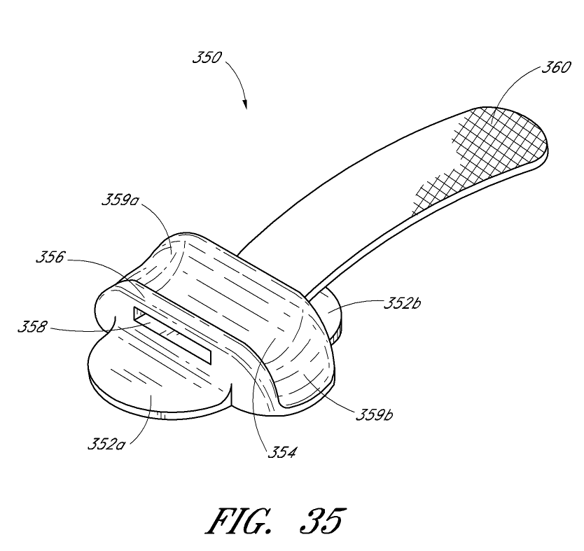

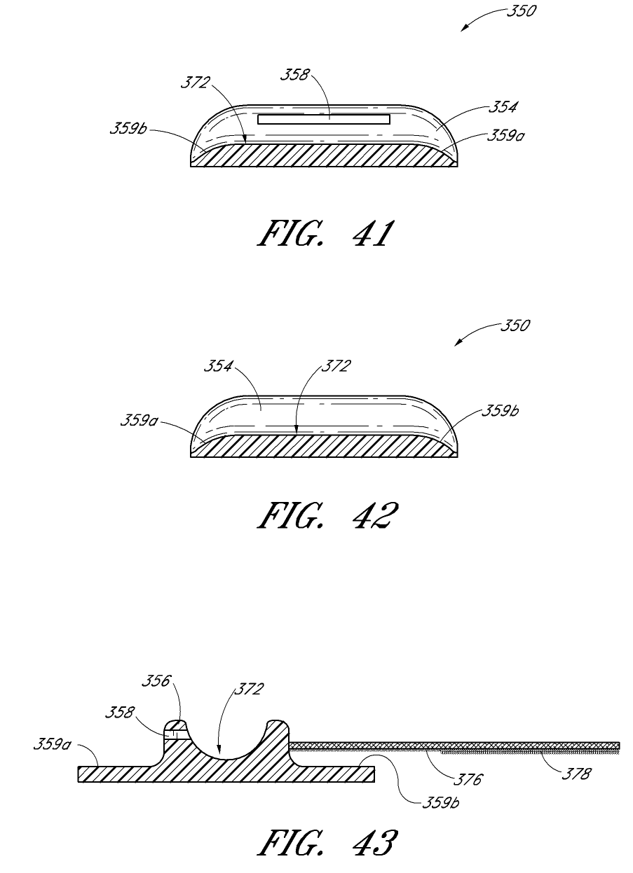

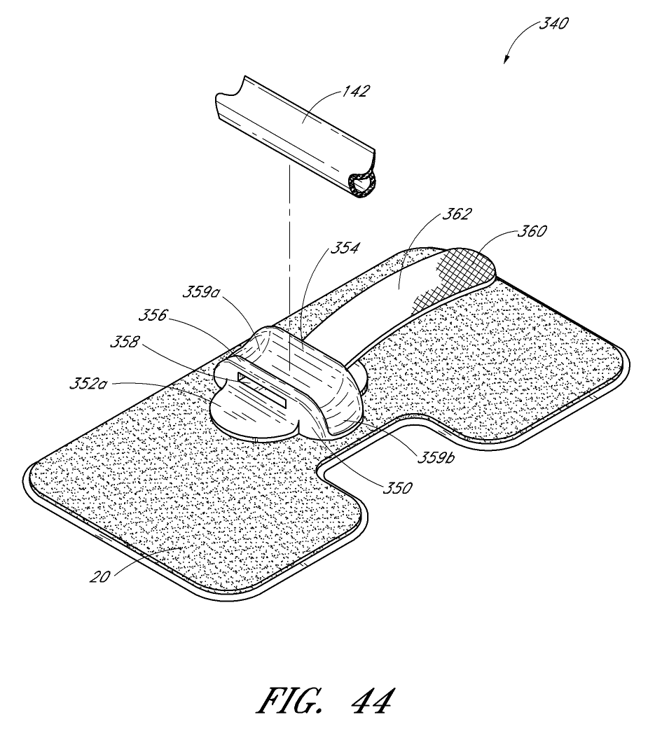

FIGS. 35-43 illustrate the retainer 350 and integral strap 360. As can be seen in a perspective view of the retainer 350, as illustrated in FIG. 35, the retainer 350 comprises wings 352a and 352b, a channel 354 configured to accept a medical article, and the strap 360. The retainer 350 also comprises a sidewall 356 with an opening 358. The opening 358 is configured to accept the strap 360. The retainer 350 also comprises curved declines 359a and 359b. A width and/or length of the wings 352a and 352b and/or channel 354 may be selected so as to allow a medical provider to easily and naturally grip the retainer 350.

In the illustrated embodiment of FIG. 36, the strap 360 extends from the retainer 350 in a generally lateral direction and is substantially longitudinally centered on the retainer 350. The strap 360 may, however, extend in a direction angled from a lateral direction or may be located at location that is not longitudinally centered on the retainer 350. Regardless of the direction in which the strap 360 extends or the location of the strap 360 along the retainer 350, the strap 360 is configured so that when folded across the channel 354, the end of the strap 360 may be placed through the opening 358. The end of the strap 360 may be rounded to facilitate placing the end through opening 358.

The shape and construction of the strap 360 may be varied. Any number of shapes or designs of the strap 360 are possible and within the scope of this description. For example, the strap 360 may taper from an area near where the strap 360 attaches to the retainer 350 towards the end of the strap 360. Although the strap 360 is illustrated as a single piece of material, the strap 360 may also comprise several pieces of material attached together.

The strap 360 is comprised of an upper surface 362. In one embodiment, the upper surface 362 comprises a material or coating that provides a high level of friction between the strap 360 and a medical article contacting the strap 360, as described in reference to the strap 40, illustrated in FIG. 10. In another embodiment, the upper surface 362 comprises a material or coating that provides a low level of friction between the strap 360 and a medical article contacting the strap 360, also as described in reference to the strap 40.

The strap 360 also comprises a hook portion 376 and a loop portion 378. In one embodiment, the hook portion 376 and loop portion 378 are attached onto a lower surface of the strap 360, as illustrated in FIG. 12. The lower surface may be integral to the upper surface 362, illustrated in FIG. 36, or the lower surface may be separate from and stacked on or laminated to the upper surface 362. In another embodiment, the hook portion 376 and loop portion 378 may be attached directly to the upper surface 362, or a surface of the book portion 376 and/or loop portion 378 may form at least part of the upper surface 362.

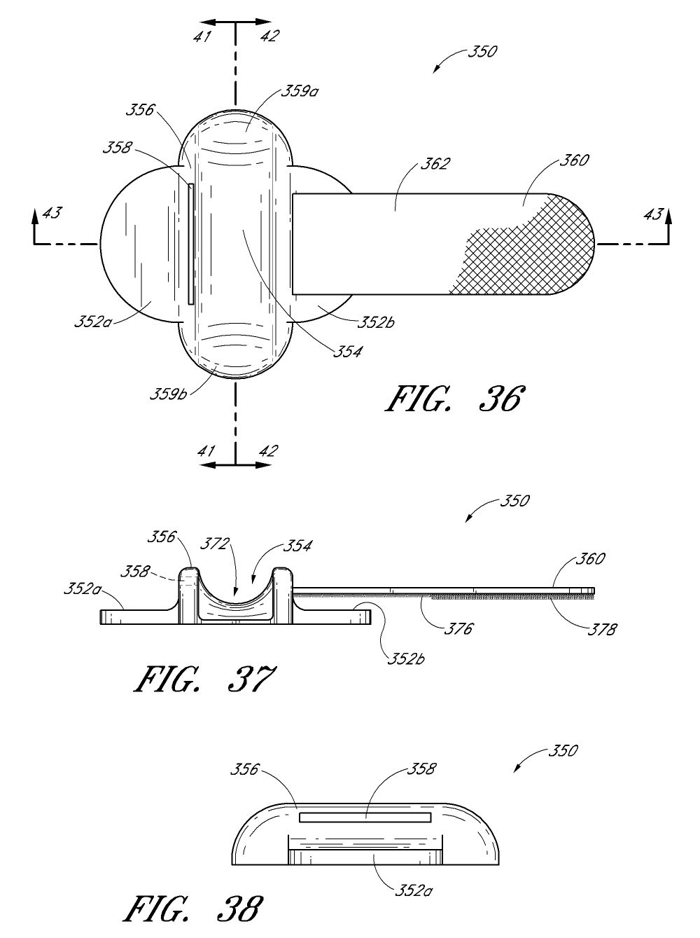

The hook portion 376 and loop portion 378 are situated next to each other. Either the hook portion 376 or loop portion 378 may be located next to the retainer 350. FIG. 37 illustrates the hook portion 376 as being located next to the retainer 350, but the placement of the hook portion 376 and loop portion 378 could be reversed, with the loop portion 378 being located next to the retainer 350. The hook portion 376 and loop portion 378 may directly abut each other and/or the retainer 350, as illustrated in FIGS. 37 and 40, or there may be a distance between the portions and/or the retainer 350.

In one embodiment, the hook portion 376 and/or loop portion 378 span the entire width of the strap 360. In another embodiment, the hook portion 376 and/or loop portion 378 may be configured as various shapes or may only partially cover the width of the strap 360.

The hook portion 376 and loop portion 378 are configured such that when the strap 360 is drawn across a medical article placed in the retainer 350, at least part of the loop portion 378 can be passed through the opening 358 and folded back to contact at least a part of the hook portion 376. More details regarding this procedure of attaching a medical article to the retainer 350 will be provided below.

The wings 352a and 352b provide a greater contact area between the retainer 350 and the anchor pad 20 than the channel 354 would provide alone, allowing for a more secure attachment. The benefits and shape of the wings 352a and 352b are similar to those of the wings 32a and 32b, described in reference to FIG. 3. The shape of the wings 352a and 352b can be seen in a top and bottom view of the retainer 350, illustrated in FIGS. 36 and 40, respectively.

In the illustrated embodiment of FIG. 38, an upper surface of the wing 352a is disposed so as to allow access to the opening 358. This disposition of the wing 352a provides space for a medical provider to place a strap through the opening 358. There exists a gap between the wing 352a and the opening 358. Portions of the strap 360 can occupy this gap before or after being inserted through the opening 358, as can the medical provider's fingers while inserting or removing the strap 360 from the opening 358.

As can be seen in FIG. 37, the channel 354 comprises a concave surface 372 (which may also be described as a groove), and is configured to accept a medical article. The channel 354 is shaped such that a medical article can be placed therein, thereby resting at least partially within the channel 354. When the medical article is placed in the channel 354, the medical article may rest on the concave surface 372. The channel 354 can be shaped in any way that allows the medical article to rest at least partially therein. In addition, the longitudinal dimension of the channel 354 may be sufficiently long to provide stability to the medical article along its length.

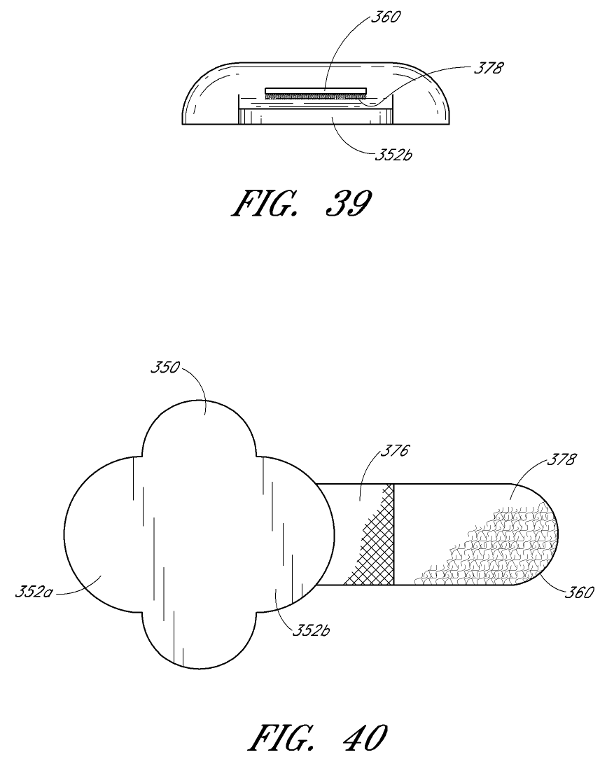

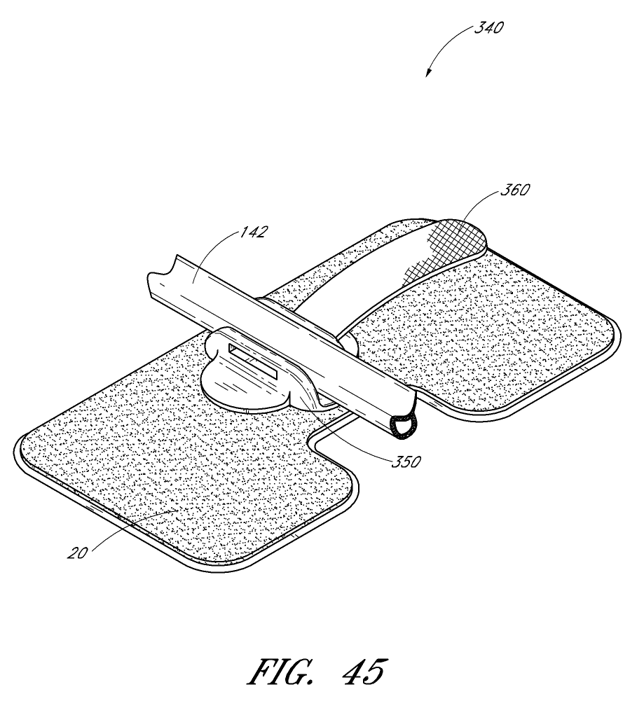

The sidewall 356 contains an opening 358. The size and shape of the opening 358 allows the strap 360 to pass through the sidewall 356. As can be seen in a cross-section taken along line 43-43 of FIG. 36, as is illustrated in FIG. 43, the opening 358 passes completely through the sidewall 356. Thus, the strap 360 can pass from the center of the retainer 350, near the concave surface 372, through the opening 358 to emerge from the side of the retainer 350 near the wing 352a. In the illustrated embodiment, the size and shape of the opening 358 roughly corresponds to a cross-section of the strap 360. The size and shape of the opening 358 can be seen in more detail in a side view of the retainer 350 taken from the side nearest which the retainer is viewed from in FIG. 35, as illustrated in FIG. 38. A size and shape of a cross-section of the strap 360 can be seen in a side view of the retainer 350 taken from the side opposite which the retainer is viewed from in FIG. 35, as illustrated in FIG. 39. Matching a cross-section of the strap 360 to the size and shape of the opening 358 minimizes longitudinal movement of the strap 360 when placed through the sidewall 356.

In one embodiment, the opening 358 is located at a similar longitudinal location along the retainer 350 as where the strap 360 is attached to the retainer 350. Thus, when the strap 360 is placed through the opening 358, the strap 360 will cross the channel 354 in a generally lateral direction. In another embodiment, the opening 358 may be located at a different longitudinal location along the retainer 350. In this embodiment, the strap will cross the channel 354 in a direction angled from a lateral direction when placed through the opening 358. The channel 354 also comprises curved declines 359a and 359b.

A medical article 142 can be placed in the retainer 350, as shown in FIGS. 44 and 45. The medical article 142 is placed above the anchoring system 340, as seen in FIG. 44. The medical provider can then lower the medical article 142 into the retainer 350. As seen in FIG. 45, the medical article 142 rests at least partially within the channel 354.

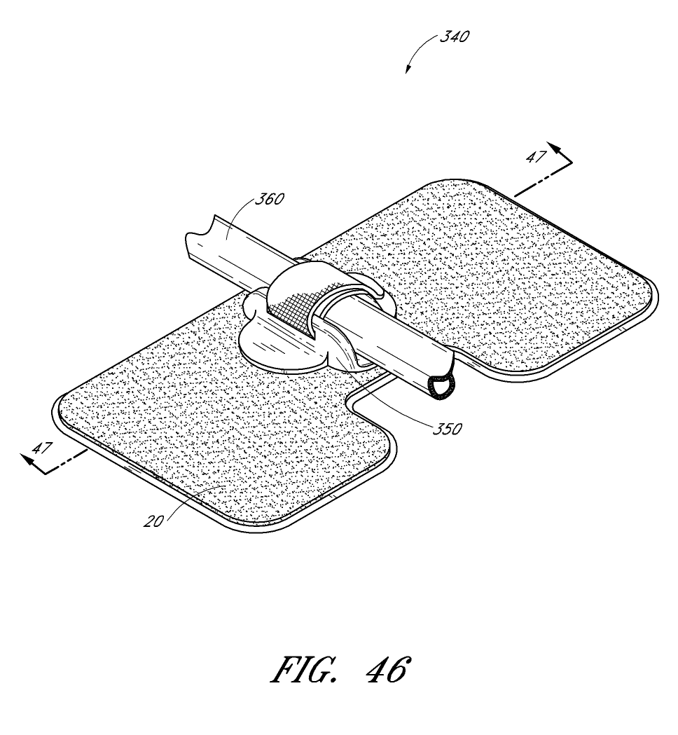

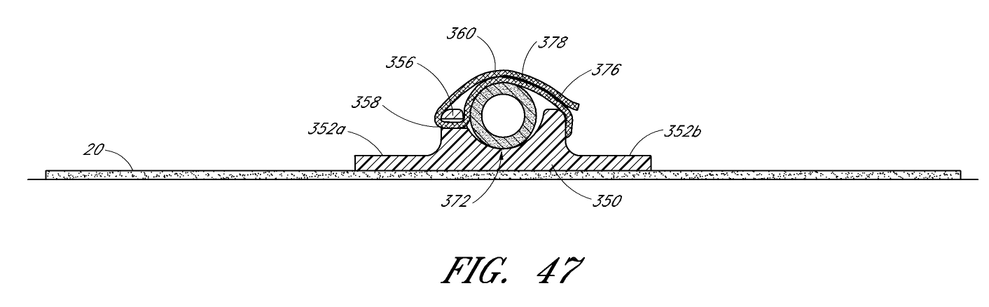

To secure the medical article 142 to the retainer 350, the medical provider places the end of the strap 360 across the medical article 142 and introduces the end into the opening 358. The medical provider draws the end through the opening 358, causing the upper surface 362 to contact the medical article 142. The medical provider then folds the strap 360 back in the opposite direction, pulling the strap 360 back towards the medical article 142 and causing the strap 360 to contact itself, as can be seen in FIG. 46. In this way, the medical article 142 can be secured to the retainer 350 and the anchoring system 340.

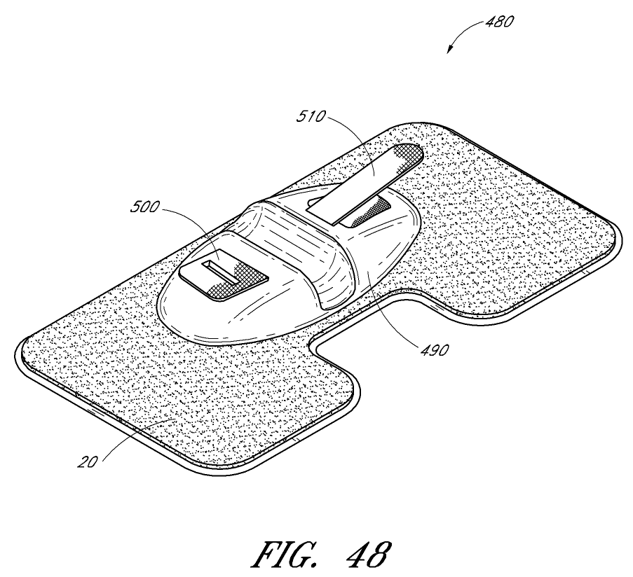

As can best be seen in FIG. 47, the strap 360 in the illustrated embodiment passes over the top of the retainer 350 and the medical article 142, through the opening 358, and then back over the top of the retainer 350 and the medical article 142. Thus, the strap 360 wraps over the top of the medical article 142 by passing through the retainer 350.

When the strap 360 is folded back to contact itself, the hook portion 376 and loop portion 378 contact and attach to each other. This increases the likelihood that the strap 360 will stay in the position in which it has been placed by the medical provider. If the strap 360 has been drawn tightly across the medical article 142, the strap 360 will hold the medical article 142 substantially in place on the retainer 350. Therefore, the strap 360 in combination with the retainer 350 will inhibit movement of the medical article 142. In this way, the anchoring system 340 can be used to secure the medical article 142 on a patient's body.

Those of skill in the art will appreciate that attaching the medical article 142 to the retainer 350 and the anchoring system 340 in this way allows the use of medical articles of varying sizes. The end of the strap 360 can be pulled through the opening 358 in varying lengths before folding the strap 360 back to contact itself. Thus, the size of an enclosed area created by the strap 360 and the channel 354 can be increased or decreased as required. The size and location of the hook portion 376 and/or the loop portion 378 may be selected to facilitate the possibility of securing medical articles of varying size to the retainer 350.

The medical provider may separate the hook portion 376 from the loop portion 378 by pulling the end of the strap 360 opposite the opening 358. When sufficient force is applied to the end of the strap 360, the hook portion 376 and loop portion 378 will detach. After the detachment, the strap 360 may be loosened about the medical article 142 or the end of the strap 360 may be removed from the opening 358. This may allow the medical article 142 to be removed from the retainer 350. Such detachment, however, will not substantially impair the hook portion 376 and loop portion 378 from reattaching at a later time. Thus, the medical article 142 or a different catheter or device may be reattached to the retainer 350 at a later time. The advantages of being able to remove a medical article and reattach the same or a different medical article have already been described above.

With reference now to FIG. 48, another embodiment of an anchoring system 480 includes an anchor pad 20, a retainer 490, a first strap 500, and a second strap 510. The retainer 490 is attached to an upper surface of the anchor pad 20. The anchor pad 20 may in turn be secured to a patient's skin. The first strap 500 and second strap 510 are attached to opposite sides of the retainer 490. The first strap 500 and second strap 510 are moveable between open and closed positions.

FIGS. 49-57 illustrate the retainer 490, the first strap 500, and the second strap 510. The retainer 490 comprises wings 492a and 492b, a channel 494 configured to accept a medical article, and a hook portion 496. The retainer 490 also comprises curved declines 499a and 499b. A width and/or length of the wings 492a and 492b and/or channel 494 may be selected so as to allow a medical provider to easily and naturally grip the retainer 490.

The first strap 500 includes an opening 504. The opening 504 is configured to receive at least a portion of the second strap 510. In the illustrated embodiment, the size and shape of the opening 504 roughly corresponds to a cross-section of the second strap 510. A size and shape of the opening 504 can be seen in a cross-section taken along the line 57-57 of FIG. 50, as illustrated in FIG. 57. A size and shape of a cross-section of the second strap 510 can be seen in a side view of the retainer 490 taken from the side opposite which the retainer is viewed from in FIG. 49, as illustrated in Figure 53. Matching a cross-section of the second strap 510 to the size and shape of the opening 504 minimizes longitudinal movement of the second strap 510 when placed through the opening 504.

The strap 500 thus has a width W5 which is wider than a width W6 of the second strap 510. The shape and construction of the first strap 500 may otherwise be varied. Any number of shapes or designs of the first strap 500 are possible and within the scope of this description. Although the first strap 500 is illustrated as a single piece of material, the first strap 500 may also comprise several pieces of material attached together.

In some embodiments, the first strap 500 may comprise a material or coating that provides a high level of friction between the first strap 500 and a medical article contacting the first strap 500. In another embodiment, the first strap 500 may comprise a material or coating that provides a low level of friction between the first strap 500 and a medical article contacting the first strap 500.

In the illustrated embodiment of FIG. 50, the second strap 510 extends from the retainer 490 in a generally lateral direction and is substantially longitudinally centered on the retainer 490. The second strap 510 may, however, extend in a direction angled from a lateral direction or may be located at location that is not longitudinally centered on the retainer 490. Regardless of the direction in which the second strap 510 extends or the location of the second strap 510 along the retainer 490, the second strap 510 is configured so that when folded across the channel 494, the end of the second strap 510 may be placed through the opening 504 of the first strap 500. The end of the second strap 510 may be rounded to facilitate placing the end through opening 504.

The shape and construction of the second strap 510 may be varied. Any number of shapes or designs of the second strap 510 are possible and within the scope of this description. For example, the second strap 510 may taper from an area near where the second strap 510 attaches to the retainer 490 towards the end of the second strap 510. Although the second strap 510 is illustrated as a single piece of material, the second strap 510 may also comprise several pieces of material attached together.

The second strap 510 includes an upper surface 512. In one embodiment, the upper surface 512 comprises a material or coating that provides a high level of friction between the second strap 510 and a medical article contacting the second strap 510. In another embodiment, the upper surface 512 comprises a material or coating that provides a low level of friction between the second strap 510 and a medical article contacting the second strap 510.

In the illustrated embodiment, the second strap 510 is attached to the retainer 490 at a similar longitudinal location as where the first strap 500 is attached to the retainer 490. Thus, when the second strap 510 is placed through the opening 504, the second strap 510 will cross the channel 494 in a generally lateral direction. In another embodiment, the second strap 510 may be attached to the retainer 490 at a longitudinal location different than where the first strap 500 is attached to the retainer 490. In this embodiment, the strap will cross the channel 494 in a direction angled from a lateral direction when placed through the opening 504.

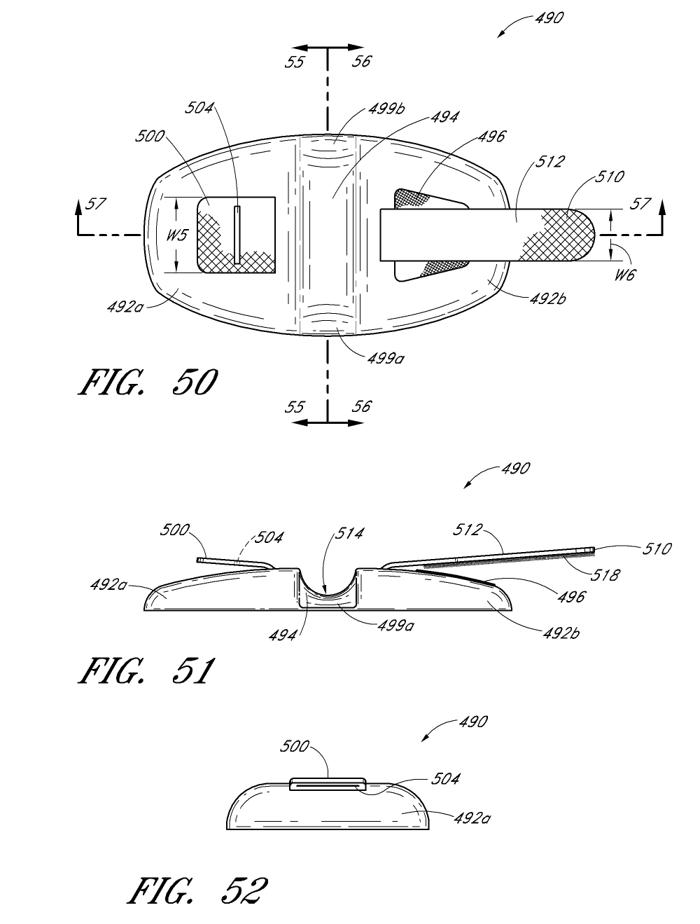

The second strap 510 also comprises a loop portion 518. In one embodiment, the loop portion 518 is attached onto a lower surface of the second strap 510, as illustrated in FIG. 51. The lower surface may be integral to the upper surface 512, illustrated in FIG. 50, or the lower surface may be separate from and stacked on or laminated to the upper surface 512. In another embodiment, the loop portion 518 may be attached directly to the upper surface 512, or a surface of the loop portion 518 may form at least part of the upper surface 512.

In one embodiment, the loop portion 518 spans the entire width W6 of the second strap 510. In another embodiment, the loop portion 518 may be configured as various shapes or may only partially cover the width W6 of the second strap 510. The loop portion 518 may additionally be configured to span varying lengths of the second strap 510. The loop portion may extend from where the second strap 510 is attached to the retainer 490 to the end of the second strap 510, or the loop portion 518 may only cover a portion of that area.

The loop portion 518 is configured such that when the second strap 510 is drawn across a medical article placed in the retainer 490, at least part of the loop portion 518 can be passed through the opening 504 of the first strap 500 and folded back to contact at least a part of the hook portion 496. More details regarding this procedure of attaching a medical article to the retainer 490 will be provided below.

The hook portion 496 is disposed on the wing 492b. It is configured to contact the loop portion 518 when the second strap 510 is folded back as described above. The hook portion 496 may otherwise be configured in any number of sizes or shapes. Alternatively, the hook portion 496 may be disposed on the second strap 510 and the loop portion may be disposed on the wing 492b.

A hook portion or a loop portion may similarly be disposed on the wing 32b of the retainer 30, the wing 192b of the retainer 190, or the wing 352b of the retainer 350, illustrated in FIGS. 3, 19, and 35, respectively. Such hook portion or loop portion may be in addition to or in place of a respective hook or loop portion disposed on the strap 40, the strap 200, or the strap 360, respectively.

The wings 492a and 492b are illustrated as having a shape that gradually slopes from the top of the retainer 490 to the base of the retainer 490, as can be seen in FIG. 51. Thus, an upper surface of the wings 492a and 492b is located closer to the top of the retainer 490 than an upper surface of the wings 32a and 32b of retainer 30, 192a and 192b of retainer 190, and 352a and 352b of retainer 350. Selecting a location of an upper surface of the wing 192b may ease attachment of the second strap 510 to the hook portion 496.

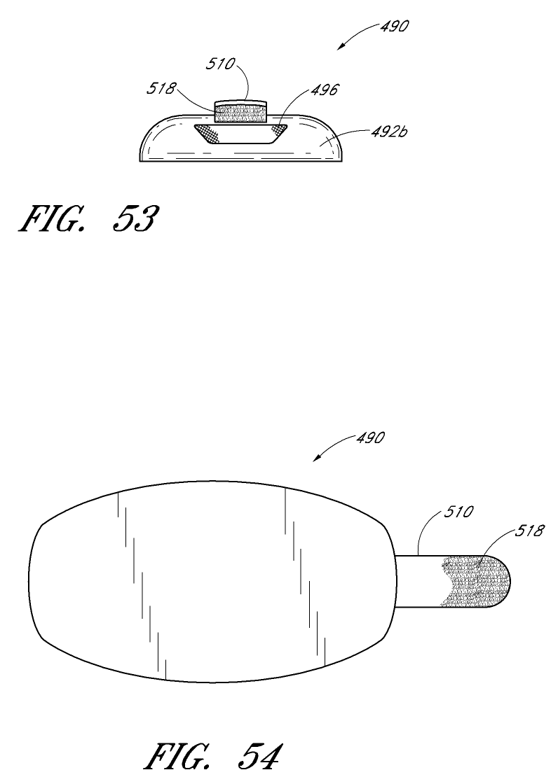

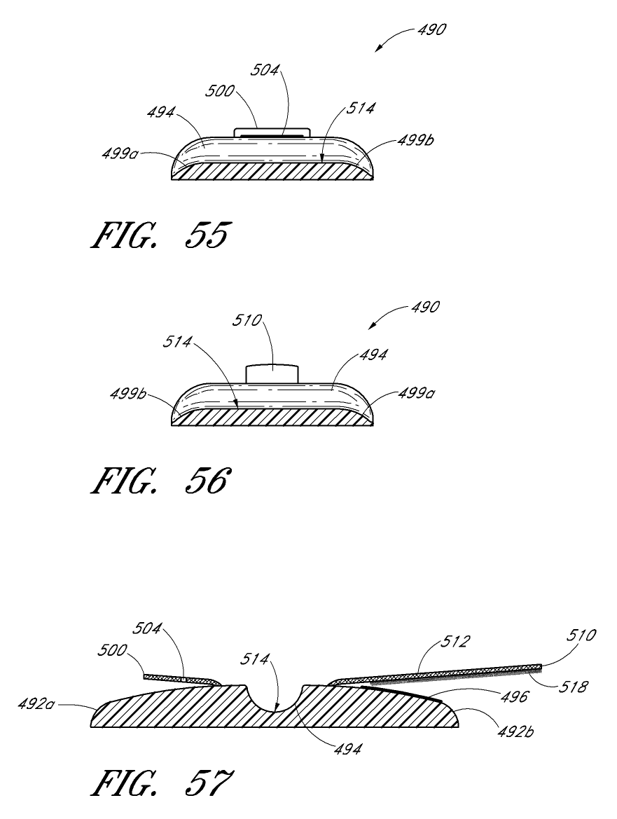

The wings 492a and 492b are additionally illustrated as extending farther from the channel 494 than the wings 32a and 32b extend from the channel 34, the wings 192a and 192b extend from the channel 194, and the wings 352a and 352b extend from the channel 354. Accordingly, the wings 492a and 492b are not semicircular in shape. The shape of the wings 492a and 492b in the current embodiment can be seen in more detail in a top view of the retainer 490, illustrated in FIG. 50; a bottom view of the retainer 490, illustrated in FIG. 54; and side views of the retainer, illustrated in FIGS. 52 and 53. The shape of the wings 492a and 492b may be selected to increase the security of attachment between the retainer 490 and the anchor pad 20, to ease manufacturing of the retainer 490, to reduce the cost of manufacturing the retainer 490, or to provide a suitable area on which to attach the hook portion 496.

As a result of the above differences between the wings 492a and 492b and the wings 32a and 32b of retainer 30, 192a and 192b of retainer 190, and 352a and 352b of retainer 350, the wings 492a and 492b are larger in size than the other described wings. Consequently, an upper surface of the wing 492b may provide more surface area on which to locate the hook portion 496 and to subsequently attach the second strap 510. In addition, the wings 492a and 492b provide a greater contact area between the retainer 490 and the anchor pad 20 than the other described wings. To add to this, the mass of the wings 492a and 492b may be increased when the size is increased, which may also provide for additional stability of the retainer 490.

The wings 492a and 492b can be adapted for use with the retainer 30, the retainer 190, or the retainer 350. Such adaption may comprise providing or forming an opening in one or both of the wings 492a and 492b and/or removing the first strap 500 and/or the second strap 510. These adaptations and other adaptations are possible and within the scope of this description.

As can be seen in FIG. 51, the channel 494 comprises a concave surface 514 (which may also be described as a groove), and is configured to accept a medical article. The channel 494 is shaped such that a medical article can be placed therein, thereby resting at least partially within the retainer 490. When the medical article is placed in the channel 494, the medical article may rest on the concave surface 514. The channel 494 can be shaped in any way that allows the medical article to rest at least partially therein. In addition, the longitudinal dimension of the channel 354 may be sufficiently long to provide stability to the medical article along its length. The channel 494 also comprises curved declines 499a and 499b.

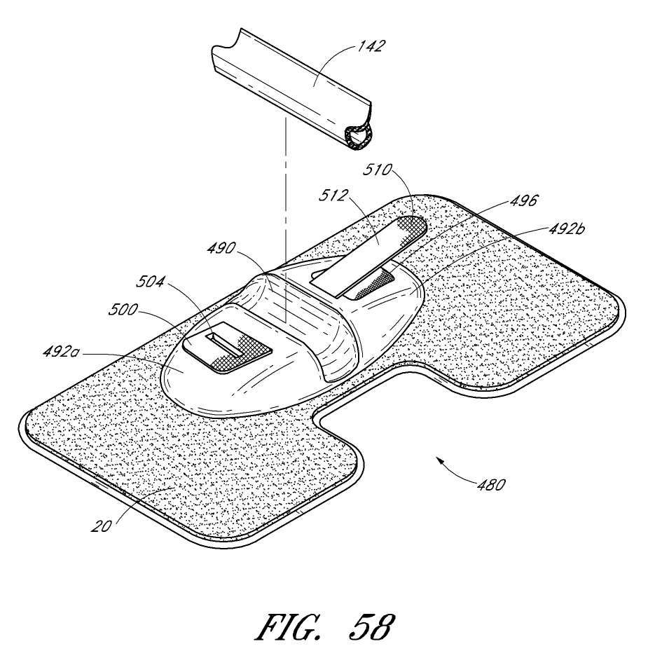

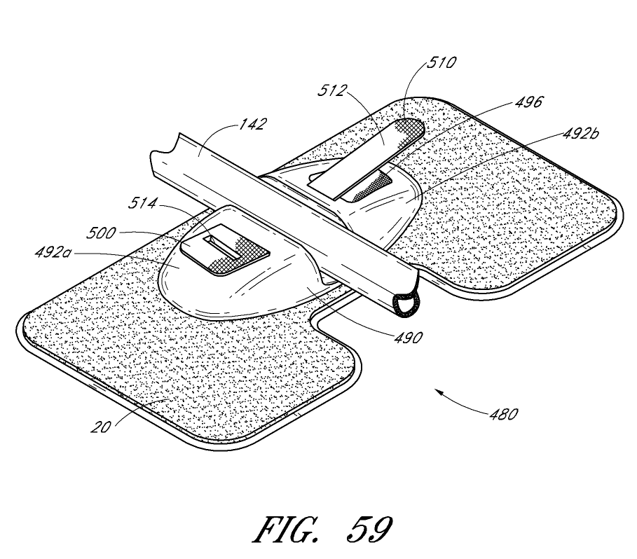

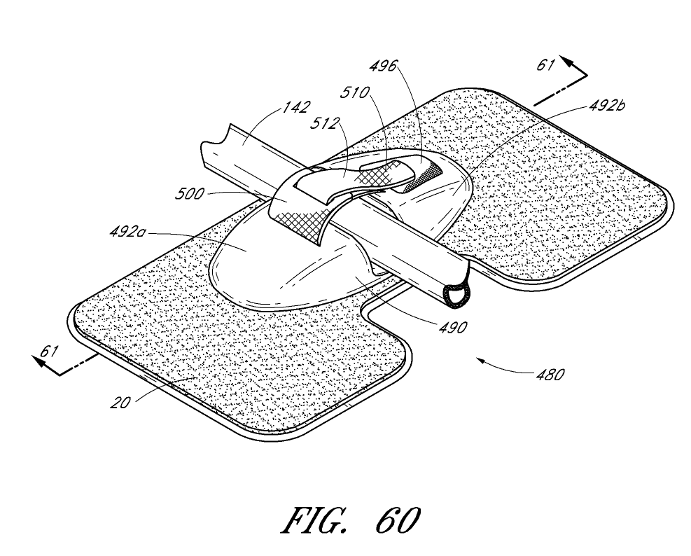

A medical article 142 can be placed in the retainer 490, as shown in FIGS. 58 and 59. The medical article 142 is placed above the anchoring system 480, as seen in FIG. 55. The medical provider can then lower the medical article 142 into the retainer 490. As seen in FIG. 59, the medical article 142 rests at least partially within the channel 494.

To secure the medical article 142 to the retainer 490, the medical provider places the end of the second strap 510 across the medical article 142, and introduces the end into the opening 504 of the first strap 500. The medical provider draws the end through the opening 504, causing the upper surface 512 of the second strap 510 to contact the medical article 142. The medical provider then folds the second strap 510 back in the opposite direction, pulling the second strap 510 back towards the medical article 142 and causing the second strap 510 to contact the wing 492b. In this way, the medical article 142 can be secured to the retainer 490 and the anchoring system 480.

As can best be seen in FIG. 61, the second strap 510 in the illustrated embodiment passes over the top of the medical article 142, through the opening 504 in the first strap 500, and then back over the top of the medical article 142. Thus, the first strap 500 and the second strap 510 wrap over the medical article 142, thereby securing the medical article 142 to the retainer 490.

When the second strap 510 is folded back to contact the wing 492b, the hook portion 496 and loop portion 518 contact and attach to each other. This increases the likelihood that the first strap 500 and the second strap 510 will stay in the positions in which they have been placed by the medical provider. If the first strap 500 and second strap 510 have been drawn tightly across the medical article 142, the first strap 500 and second strap 510 will hold the medical article 142 substantially in place on the retainer 490. Therefore, the first strap 500 and the second strap 510 in combination with the retainer 490 will inhibit movement of the medical article 142. In this way, the anchoring system 480 can be used to secure the medical article 142 on a patient's body.

Those of skill in the art will appreciate that attaching the medical article 142 to the retainer 490 and the anchoring system 480 in this way allows the use of medical articles of varying sizes. The end of the second strap 510 can be pulled through the opening 504 in the first strap 500 in varying lengths before folding the second strap 510 back to contact the wing 492b. Thus, the size of an enclosed area created by the first strap 500, the second strap 510, and the channel 494 can be increased or decreased as required. The size and location of the hook portion 496 and/or the loop portion 518 may be selected to facilitate the possibility of securing medical articles of varying size to the retainer 480.

The medical provider may separate the hook portion 496 from the loop portion 518 by pulling the end of the second strap 510 away from the wing 492b. When sufficient force is applied to the end of the second strap 510, the hook portion 496 and loop portion 518 will detach. After the detachment, the first strap 500 and the second strap 510 may be loosened about the medical article 142 or the end of the second strap 510 may be removed from the opening 504 of the first strap 500. This may allow the medical article 142 to be removed from the retainer 490. Such detachment, however, will not substantially impair the hook portion 496 and loop portion 518 from reattaching at a later time. Thus, the medical article 142 or a different catheter or device may be reattached to the retainer 490 at a later time. The advantages of being able to remove a medical article and reattach the same or a different medical article have already been described above.

With reference now to FIG. 62, another embodiment of an anchoring system 620 includes an anchor pad 622, a strap 624, and a retainer 630. The retainer 630 comprises a channel 634 configured to accept a medical article. The anchoring system 620 is similar to the anchoring system 10 illustrated in FIG. 1, except the openings 38a and 38b in the retainer 30 have been omitted in favor of a single opening 632 forming a passageway through the retainer 630. The passageway is disposed under a concave surface 636 of the channel 634. The opening 632 and/or the passageway may or may not be parallel to the base or angled relative to the lateral, longitudinal, and transverse directions. In addition, one or more portions of the opening 632 and/or the passageway may be angled relative to another portion of the opening 632 or passageway. In the illustrated embodiment, the opening 632 is substantially parallel to the base of the retainer 630, as can be seen in a cross-sectional view of the retainer 630, illustrated in FIG. 63.

An adhesive spot 638 may be advantageously disposed upon the channel 634. In addition or in the alternative, an adhesive spot may be disposed upon the strap 624 or another structure that contacts the medical article. This adhesive spot may take the form of a glue dot, as is illustrated in FIGS. 62 and 63. Such glue dots are desirably formed of a material which exhibits high resistance to shear and which can be peeled off of a medical article without leaving a residue. Such an adhesive is sold by All-Pak Inc. of New Berlin, Wis. as part number GD-06 “Super High Tack Glue Dot.”

Although the adhesive spot 638 is illustrated as a single glue dot on the channel 634, multiple glue dots may be used. It is not necessary, however, for multiple glue dots to be used; a single glue dot disposed upon the channel 634 may advantageously be used to provide greater frictional and transverse forces between the retainer 630 and a medical article.

Furthermore, the adhesive spot 638 need not be a single point of adhesive. In further preferred designs the adhesive spot may be a region composed of an elastic and compressively deformable material such Kraton® polymer compounds. Such a compound includes Dynaflex® G2706 available from GLS Corporation, as well as other thermoplastic elastomers or silicone or urethane epoxies.

This region also need not be round. In further preferred designs, a large region of the surface of the channel 634 may be covered with a suitable material, such as Kraton®. For instance, the entire surface of the channel 634 might be covered with a thin layer of adhesive to advantageously provide additional traction and transverse bias between a medical article and the retainer 630.

Other means of producing an appropriate adhesive spot for use with various preferred embodiments of the present invention include without limitation: treating a portion of the surface of the channel 634 chemically or electrically to adjust its surface friction or compressibility; spraying or spreading an adhesive coating onto a portion of the channel 634; attaching peel-off adhesive members to portions of the channel 634; injection molding regions of adhesive or compressible material, such as Kraton®, to a portion of the surface of the channel 634; or such other means as are known in the art.

The adhesive spot 638 may also be implemented on the retainers 30, 190, 350, and 490, previously illustrated in FIGS. 3, 19, 35, and 49, respectively. An adhesive spot may additionally or alternatively be implemented on other structure that contacts the medical article in the anchoring systems 10, 180, 340, and 480, illustrated in FIGS. 1, 18, 34, and 48, respectively. The adhesive spot 638 may likewise be omitted from the retainer 630.

The anchoring system 620 may otherwise be configured similar to the anchoring system 10 illustrated in FIG. 1. The anchor pad 622 may be the same as the anchor pad 20 illustrated in FIG. 1 and is configured to support the retainer 630. The strap 624 may be configured the same as the strap 40 illustrated in FIG. 1, although the strap 624 may be longer than the strap 20 such that the strap 624 can be passed through the opening 632, around the portion of the retainer above the opening 632, and around a medical article. The retainer 630 may otherwise be configured the same as the retainer 30.

The anchoring system 620 can be assembled by appropriately attaching or reattaching the strap 624 to the retainer 630, which is attached to the anchor pad 622. Preferably, the retainer 630, the anchor pad 622, and the strap 624 are packaged and delivered to the medical provider in an assembled state. Of course the medical provider could assemble one or more of the components of the anchoring system 620. For example, the retainer 630 and the strap 624 could be delivered in an unassembled state to the medical provider. The medical provider could then assemble the strap 624 to the retainer 630.

To attach the strap 624 to the retainer 630, a manufacturer or medical provider introduces either end of the strap 624 into one side of the opening 632. The strap 624 can be pushed through the opening 632 until the end of the strap 624 emerges from the other side of the opening 632. In this way, the strap 624 will pass from one side of the retainer 630, through the opening 632 and under the concave surface 636, to the other side of the retainer 630, thereby attaching to the retainer 630, as shown in FIG. 62. The strap 624 thus is situated in a generally lateral direction, although other embodiments may include selecting the configuration of the opening 632 and attaching the strap 624 to the retainer 630 so that the strap 624 is angled from a lateral, longitudinal, and/or transverse direction.

To secure a medical article to the retainer 630, a medical article is placed in the retainer 630. The medical article is initially placed above the anchoring system 620, and can then be lowered into the retainer 630 to rest at least partially within the channel 634. The medical article may be placed within the channel 634 either before or after the strap 624 is attached to the retainer 630. Once the strap 624 is attached to the retainer 630 and the medical article is placed within the channel 634, the strap 624 may be wrapped around the medical article to secure the medical article to the retainer 630 as described above in reference to FIGS. 15 and 16. In this way, the anchoring system 620 can be used to secure a medical article on a patient's body.

Attaching a medical article to the retainer 630 and the anchoring system 620 in this way allows the use of medical articles of varying sizes, and allows removal of the medical article from the anchoring system 620, as described above in reference to FIG. 17.

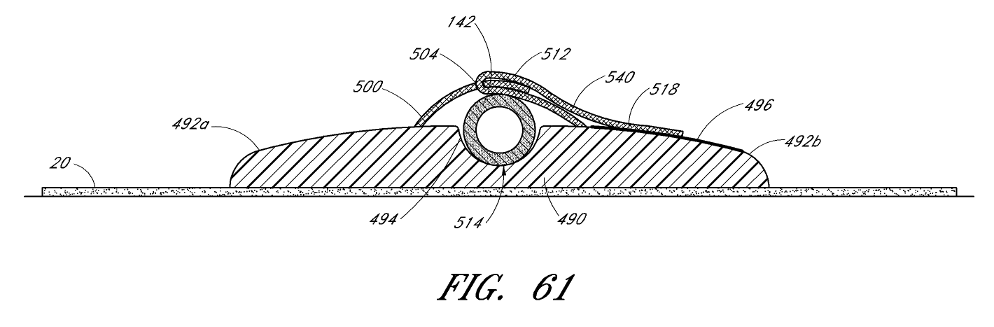

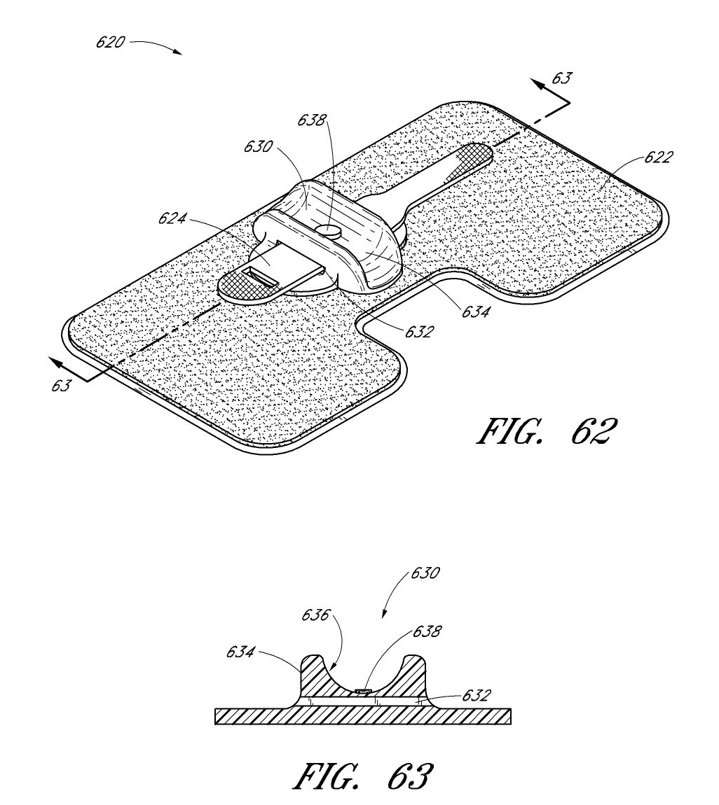

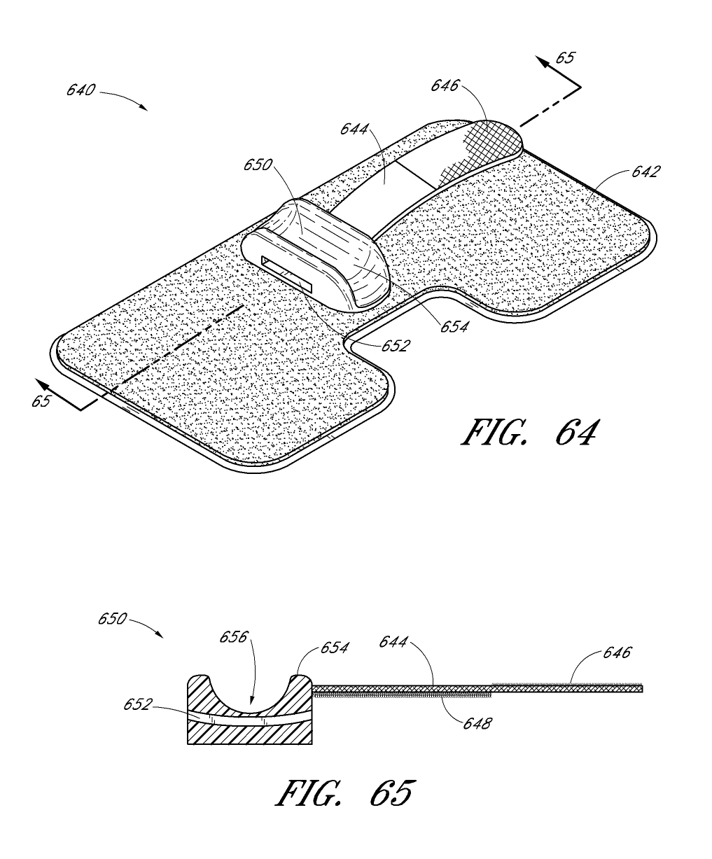

With reference now to FIG. 64, another embodiment of an anchoring system 640 includes an anchor pad 642, a strap 644, and a retainer 650. The strap 644 is integral with the retainer 630. The retainer 630 comprises a channel 654 configured to accept a medical article. The anchoring system 640 is similar to the anchoring system 340 illustrated in FIG. 34, except that the wings 352a and 352b of the retainer 350 have been omitted from the retainer 650, and the opening 358 has been omitted in favor of an opening 652 forming a passageway through the retainer 650. The passageway is disposed under a concave surface 656 of the channel 654.

Omitting wings from the retainer 650 may increase the ease with which a medical provider can cause the strap 644 to pass through the opening 652. Of course, the retainer 630 may alternatively comprise one or more wings. Likewise, the retainers 30, 190, 350, and 630, previously illustrated in FIGS. 3, 19, 35, and 63, respectively, may omit wings.

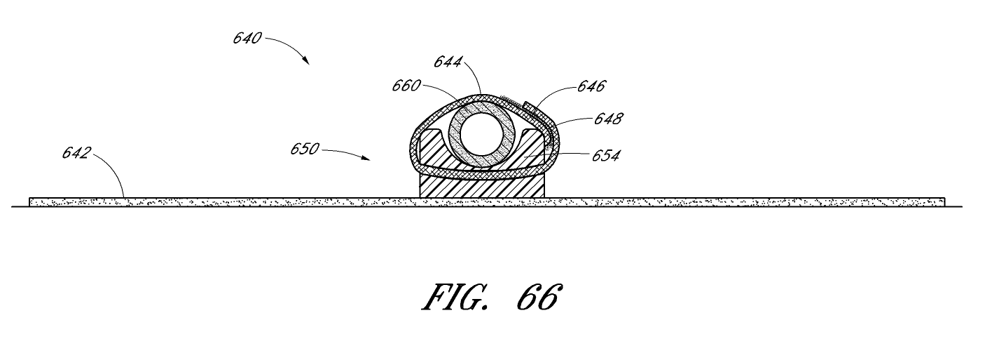

FIG. 65 illustrates a cross-sectional view of the retainer 650 and integral strap 644. The opening 652 and the passageway in the retainer 350 differs from the opening 632 and the passageway in the retainer 630 in that a cross-section of the opening 652 and the passageway is generally curved. This general curvature can be selected to be similar to the curvature of the concave surface 656. Selecting the curvature in this way may decrease the length of the strap 644 necessary to attach a medical article to the anchoring system 640 and may increase the security with which the medical article is attached. Alternatively, a cross-section of the opening 652 and the passageway may be selected to be of a different shape. The opening 652 and/or the passageway may be configured similar to the opening 632 and the passageway in the retainer 630. Likewise, the opening 632 and/or the passageway in the retainer 630 may configured similar to the opening 652 and the passageway in the retainer 650.

The strap 644 comprises a hook portion and a loop portion disposed on opposite sides of the strap 644 and configured to attach to each other. In the illustrated embodiment, the strap 644 comprises a hook portion 646 on a top side thereof, and further comprises a loop portion 648 on the bottom side thereof. Of course, the hook portion 646 may be disposed on the bottom side of the strap 644 and the loop portion 648 may be disposed on the top surface of the strap 644. In the illustrated embodiment, each of the hook portion 646 and the loop portion 648 partially cover the surface on which it is disposed, but in other embodiments one or both of the hook portion 646 and the loop portion 648 may cover the entirety of the surface on which it is disposed.

The anchoring system 640 may otherwise be configured similar to the anchoring system 340 illustrated in FIG. 34. For example, the anchor pad 642 may be the same as the anchor pad 20 illustrated in FIG. 1 and is configured to support the retainer 650. The retainer 650 may otherwise be configured the same as the retainer 350, and the strap 644 may otherwise be configured the same as the strap 360.

To secure a medical article 660 to the retainer 650, the medical article 660 is placed in the retainer 650. The medical article 660 is initially placed above the anchoring system 640, and can then be lowered into the retainer 650 to rest at least partially within the channel 654. Once the medical article 660 is placed within the channel 654, the strap 644 may be drawn across the medical article 660 and the end of the strap 644 introduced into the opening 652 on the side of the retainer 650 opposite the side on which the strap 644 is attached to the retainer 650. The strap 644 can then be pushed through the opening 652 until the end of the strap 644 emerges from the other side of the opening 652 (i.e. the side on which the strap 644 is attached to the retainer 650). In this way, the strap 644 passes from a first side of the retainer 650, over the medical article 660 to a second side of the retainer 650, and through the opening 652 and under the concave surface 656, to return to the first side of the retainer 650. Subsequently, the end of the strap 644 can be pushed against another portion of the strap 644, thereby causing the hook portion 646 and the loop portion 648 to contact each other and attach. In this way, the strap 644 will encircle the medical article 660 and attach the medical article 660 to the anchoring system 640, as can be seen in a cross-sectional view of the anchoring system 640 and the medical article 660, illustrated in FIG. 66.

Attaching a medical article to the retainer 650 and the anchoring system 640 in this way allows the use of medical articles of varying sizes, and allows removal of the medical article from the anchoring system 640, as described above in reference to FIG. 47.

As is apparent from the foregoing description the medical article is readily releasable from the retainer and the retainer can be easily refastened and adjusted as desired. The present anchoring system thus provides a sterile, tight-gripping, needle-free and tape-free way to anchor a medical article to a patient. The retainer thus eliminates use of tape, and if prior protocol required suturing, it also eliminates accidental needle sticks, suture-wound-site infections and scarring. In addition, the retainer can be configured to be used with any of a wide variety of catheters, tubes, wires, and other medical articles. Patient comfort is also enhanced and application time is decreased with the use of the present anchoring system.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. An anchoring system for securing a medical article to a body of a patient, comprising:
an anchor pad having an upper surface and a lower surface, at least a portion of the lower surface having an adhesive surface for contacting the patient's skin;
a retainer supported by the anchor pad and comprising a channel, the channel being configured to accept a portion of the medical article; and
at least one flexible strap, the strap being configured to wrap around at least a portion of the medical article so as to releasably secure the medical article to the retainer.

2. The anchoring system of claim 1 further comprising a recess, at least a portion of said flexible strap being disposed in said recess.

3. The anchoring system of claim 2, wherein a radius of the recess is greater than a radius of the channel.

4. The anchoring system of claim 2, wherein at least a portion of the recess includes an adhesive, the adhesive being configured to permanently secure the at least strap to the retainer.

5. The anchoring system of claim 1 further comprising at least two openings, the flexible strap passing through the openings at least when the medical article is secured to the retainer.

6. The anchoring system of claim 5, wherein the at least two openings are in the retainer.

7. The anchoring system of claim 5, wherein at least one of the two openings is in the at least one strap.

8. The anchoring system of claim 1 further comprising at least three openings, the flexible strap passing through the openings at least when the medical article is secured to the retainer.

9. The anchoring system of claim 1 further comprising a hook and loop type fastener configured to secure the at least one strap to itself.

10. The anchoring system of claim 1, wherein the at least one strap and the retainer are a unitary structure.

11. A medical line securement system comprising:
a medical article;
an anchor pad including a lower adhesive surface configured to attach to an epidermal layer of a patient;
a retainer having a generally concave portion opposite the anchor pad; and
at least two opposing straps joined at a central section, the central section having a lower side adhered to the generally concave portion, and the at least two opposing straps and central section having an upper side configured to contact the medical article so as to inhibit its longitudinal movement.

12. The medical line securement system of claim 11, wherein the two opposing straps wrap around the medical article without substantial occlusion.

13. A securement device configured for use with a medical article, the device comprising:
a retainer configured to receive a medical article and having at least one opening; and
at least one strap disposed within the at least one opening and configured to releasably engage the medical article.

14. The retainer of claim 13, wherein the at least one strap is adhered to the retainer.

15. The retainer of claim 13, wherein a lower surface of the at least one strap adheres to the retainer and an upper surface of the at least one strap contacts the medical article.

16. The retainer of claim 13, wherein the at least one strap does not substantially occlude the medical article.

17. The retainer of claim 13, wherein at least two straps are configured to engage the medical article.

18. The retainer of claim 17, wherein one of the at least two straps is configured to slide within and through another of the at least two straps.

19. A method of securing a medical article to a patient, the method comprising:
providing a medical article;
providing a retainer having a channel, an opening, and at least one strap attached to said retainer;
positioning a portion of the medical article over the channel;
folding the at least one strap over the portion of the medical article such that the at least one strap covers the portion of the medical article;
threading at least a portion of the folded strap through the opening; and
releasably securing the threaded portion to another portion of the at least one strap.

20. The method of claim 19 further comprising sliding the at least one strap in a lateral direction relative to the retainer.
